(19) United States
(12) Patent Application Publication
(10) Pub. No.: US 2024/0309458 A1
(43) Pub. Date: Sep. 19, 2024

(54) MYC-HSF1 DUAL AMPLIFICATION AS A BIOMARKER FOR CANCER TREATMENT

(71) Applicant: The Trustees of Indiana University, Bloomington, IN (US)

(72) Inventor: Richard Leon Carpenter, Bloomington, IN (US)

(21) Appl. No.: 18/418,110

(22) Filed: Jan. 19, 2024

Related U.S. Application Data
(60) Provisional application No. 63/480,768, filed on Jan. 20, 2023.

(51) Int. Cl. C12Q 1/6886; A61K 31/519; A61P 35/00; G01N 33/50

(52) U.S. Cl. CPC C12Q 1/6886; A61K 31/519; A61P 35/00; G01N 33/5011; C12Q 2600/106; C12Q 2600/156

(57) ABSTRACT

Disclosed herein are methods for using dual amplification of MYC and HSF1 in cancer cells to identify cancer cells having increased susceptibility to a therapeutic inhibitor. Disclosed herein are methods for treating a patient with cancer where the cancer cells have coamplification of MYC and HSF1. Also disclosed are methods for identifying therapeutic target and therapeutic compounds by screening with cells known to have dual amplification of MYC and HSF1.

### CROSS-REFERENCE TO RELATED APPLICATION

The present application claims priority to U.S. Provisional Application No. 63/480,768, filed on Jan. 20, 2023, and entitled “MYC-HSF1 DUAL AMPLIFICATION AS A BIOMARKER FOR CANCER TREATMENT,” the entire disclosure of which is expressly incorporated by reference herein.

### STATEMENT OF GOVERNMENTAL RIGHTS

This invention was made with government support under CA207575 awarded by the National Institute of Health. The Government has certain rights in the invention.

### FIELD OF THE INVENTION

This disclosure generally relates to cancer (e.g., ovarian cancer), cancer treatments, and predictive methods for cancer treatments based on dual gene amplification. The disclosure further relates to methods of identifying cancer treatments having higher efficacy for cancers having dual gene amplification and identification of patients who may benefit from such treatments.

### BACKGROUND

Ovarian Cancer is the 5th leading cause of cancer-related death in women in the US and has a dismal prognosis. The majority of ovarian cancer patients present with tumors that have already metastasized at the time of diagnosis often due to a lack of early detection. In addition to lack of early detection, effective therapies for the majority of patients who present with metastasis are lacking. Thus, ovarian cancer has a cure rate of only 30%, with high-grade serious disease accounting for 70%-80% of ovarian cancer deaths. The complexity of ovarian cancer underlies the need for a better understanding of the molecular mechanism of this disease and the need for novel and improved therapies.

While many cancer therapeutics have been, and more continue to be, developed, it is often difficult to determine which therapeutic will have the greatest efficacy against a specific type of cancer. As these cancer therapeutics become more targeted, this difficulty increases because the effectiveness of the cancer therapy also becomes more directly linked with the genotype of the cancer cell being targeted. There is a need in cancer treatments, and particularly in ovarian cancer treatments, to identify the available cancer therapy having the greatest efficacy against the specific cancer cells of a patient. There is a need in clinical trials for cancer treatments to select the right patient population having cancer cell type to which the cancer treatment will be most effective. And there is a need for methods for identifying additional or new therapeutics for use against specific cancer cell targets. Aspects of the invention disclosed herein address these needs.

### SUMMARY OF THE INVENTION

A first aspect of the invention includes methods for determining susceptibility, such as enhanced susceptibility, of a cell to a therapeutic inhibitor.

A second aspect of the invention includes methods for screening a compound, such as an epigenetic inhibitor, against a biological sample which includes cells that have dual gene amplification of MYC and HSF1.

A third aspect of the invention includes methods of treating cancer in a subject, where the subject has cancer cells with dual amplification of MYC and HSF1 genes.

A first embodiment is a method of determining whether a biological sample obtained from a human has increased susceptibility to a therapeutic inhibitor including: measuring copy number of the MYC gene in the biological sample and determining whether or not the MYC gene has a copy number of greater than or equal to three; and measuring copy number of the HSF1 gene in the biological sample and determining whether or not the HSF1 gene has a copy number of greater than or equal to three, wherein determining the copy number of greater than or equal to three for the MYC gene and the HSF1 gene indicates increased susceptibility to the therapeutic inhibitor.

A second embodiment is a method of determining whether a biological sample obtained from a human has increased susceptibility to a therapeutic inhibitor, where the biological sample is a tumor.

A third embodiment is a method of determining whether a biological sample obtained from a human has increased susceptibility to a therapeutic inhibitor, where the biological sample is a tumor including ovarian cancer cells.

A fourth embodiment is a method of determining whether a biological sample obtained from a human has increased susceptibility to a therapeutic inhibitor, where determining a copy number of the MYC gene of greater than or equal to five and a copy number of the HSF1 gene of greater than or equal to five in at least 5% of the ovarian cancer cells in the biological sample indicates increased susceptibility to the therapeutic inhibitor.

A fifth embodiment is a method of determining whether a biological sample obtained from a human has increased susceptibility to a therapeutic inhibitor, where measuring copy number comprises analyzing the biological sample with fluorescence in situ hybridization, comparative genomic hybridization, polymerase chain reaction, next-generation sequencing, southern blot analysis, immunohistochemistry, or a combination thereof.

A sixth embodiment is a method of determining whether a biological sample obtained from a human has increased susceptibility to a therapeutic inhibitor, where the therapeutic inhibitor is a PLK1 inhibitor or an HDAC inhibitor.

A seventh embodiment is a method of determining whether a biological sample obtained from a human has increased susceptibility to a therapeutic inhibitor, where the HDAC inhibitor is entinostat, vorinostat, romidepsin, panobinostat, or belinostat; and the PLK1 inhibitor is volasertib, BI2536, BI6727, NMS-1286937, or GSK461364.

An eighth embodiment is a method for screening an epigenetic inhibitor against a biological sample including cancer cells, where at least 5% of the cancer cells comprise greater than or equal to three gene copies of MYC and greater than or equal to three gene copies of HSF1, including a) contacting the biological sample with the epigenetic inhibitor; b) measuring average cell viability of the biological sample following contact with the epigenetic inhibitor; and c) determining whether the biological sample has reduced average cell viability following contact with the epigenetic inhibitor relative to an untreated portion of the biological sample, wherein reduced average cell viability indicates increased susceptibility to the epigenetic inhibitor.

A ninth embodiment is a method for screening an epigenetic inhibitor against a biological sample including cancer cells, where average cell viability is measured using a dye exclusion assay, a colorimetric assay, a fluorometric assay, a luminometric assay, or a flow cytometric assay.

A tenth embodiment is a method for screening an epigenetic inhibitor against a biological sample including cancer cells, wherein the biological sample comprises prostate cancer cells, bladder cancer cells, breast cancer cells, ovarian cancer cells, colorectal cancer cells, lung cancer cells, or esophageal cancer cells.

An eleventh embodiment is a method for screening an epigenetic inhibitor against a biological sample including cancer cells, wherein the epigenetic inhibitor is an HDAC inhibitor.

A twelfth embodiment is a method for screening an epigenetic inhibitor against a biological sample including cancer cells, which includes contacting the biological sample with the epigenetic inhibitor and a PLK-1 inhibitor.

A thirteenth embodiment is a method for screening an epigenetic inhibitor against a biological sample including cancer cells, including contacting the biological sample with the epigenetic inhibitor and a PLK-1 inhibitor, wherein the PLK-1 inhibitor is volasertib, BI2536, BI6727, NMS-1286937, or GSK461364.

A fourteenth embodiment is a method of treating a cancer in a mammalian subject including administering a therapeutically effective amount of an inhibitor to the subject, wherein at least one cell in a sample of cancer cells obtained from the mammalian subject has greater than or equal to three gene copies of MYC and greater than or equal to three gene copies of HSF1.

A fifteenth embodiment is a method of treating a cancer in a mammalian subject, wherein the cancer is ovarian cancer, prostate cancer, bladder cancer, breast cancer, ovarian cancer, colorectal cancer, lung cancer, or esophageal cancer.

A sixteenth embodiment is a method of treating a cancer in a mammalian subject, wherein the inhibitor is a PLK1 inhibitor.

A seventeenth embodiment is a method of treating a cancer in a mammalian subject, wherein the PLK1 inhibitor is volasertib, BI2536, BI6727, NMS-1286937, or GSK461364.

An eighteenth embodiment is a method of treating a cancer in a mammalian subject, wherein the inhibitor is an HDAC inhibitor.

A nineteenth embodiment is a method of treating a cancer in a mammalian subject, wherein the HDAC inhibitor is entinostat, vorinostat, romidepsin, panobinostat, or belinostat.

A twentieth embodiment is a method of treating a cancer in a mammalian subject, where at least 5% of cells in the sample of cancer cells obtained from the subject have greater than or equal to five gene copies of MYC and greater than or equal to five gene copies of HSF1.

A fourth aspect of the invention includes methods for using dual amplification of MYC and HSF1 in cancer cells to identify cancer cells having increased susceptibility to a therapeutic inhibitor.

A fifth aspect of the invention includes methods for treating a patient with cancer where the cancer cells have coamplification of MYC and HSF1.

A sixth aspect of the invention includes methods for identifying therapeutic target and therapeutic compounds by screening with cells known to have dual amplification of MYC and HSF1.

A twenty-first embodiment is a method of treating a subject with a cancer that includes the steps of obtaining a sample of the cancer cells from the subject; determining the copy number of the MYC gene and the copy number of the HSF1 gene in the sample; comparing the copy number for both MYC and HSF1 in the sample to a control copy number to determine if a gain of copy number is present in the sample; selecting a therapeutically effective inhibitor for the subject with the gain in copy number for both MYC and HSF1 present in sample; and administering a therapeutically effective amount of the inhibitor to the subject with the gain in copy number for both MYC and HSF1.

A twenty-second embodiment is a method of treating a subject with a cancer where the cancer is ovarian cancer.

A twenty-third embodiment is a method of treating a subject with a cancer where the inhibitor administered is a PLK1 inhibitor.

A twenty-fourth embodiment is a method of treating a subject with a cancer where the inhibitor administered is volasertib.

A twenty-fifth embodiment is a method of treating a subject with a cancer where the inhibitor administered is a HDAC inhibitor.

A twenty-sixth embodiment is a method of treating a subject with a cancer where the inhibitor administered is entinostat.

A twenty-seventh embodiment is a method of treating a subject with a cancer where the gain of copy number of MYC is greater than or equal to three copies and the gain in copy number of HSF1 is greater than or equal to three copies in at least 5% of the cancer cells in the subject sample.

An twenty-eighth embodiment is a method of treating a subject with a cancer where the gain of copy number of MYC is greater than or equal to five copies and the gain in copy number of HSF1 is greater than or equal to five copies in at least 5% of the cancer cells in the subject sample.

A twenty-ninth embodiment is a method of identifying cancer cells in a subject having increased susceptibility to a therapeutic inhibitor by performing the steps of obtaining a sample of the cancer cells from the subject; and determining the copy number of the MYC gene and the copy number of the HSF1 gene in the sample; comparing the copy number for both MYC and HSF1 in the sample to a control copy number to determine if a gain of copy number is present in the sample, wherein a gain in copy number for both MYC and HSF1 represents an increased susceptibility of the cancer cell to the therapeutic inhibitor.

A thirtieth embodiment is a method of identifying cancer cells in a subject having increased susceptibility to a therapeutic inhibitor where the cancer is ovarian cancer.

A thirty-first embodiment is a method of identifying cancer cells in a subject having increased susceptibility to a therapeutic inhibitor where the therapeutic inhibitor is PLK1 inhibitor

A thirty-second embodiment is a method of identifying cancer cells in a subject having increased susceptibility to a therapeutic inhibitor where the therapeutic inhibitor is a HDAC inhibitor.

A thirty-third embodiment is a method of identifying cancer cells in a subject having increased susceptibility to a therapeutic inhibitor where the gain of copy number of MYC is greater than or equal to three copies and the gain in copy number of HSF1 is greater than or equal to three copies in at least 5% of the cancer cells in the subject sample.

A thirty-fourth embodiment is a method of identifying cancer cells in a subject having increased susceptibility to a therapeutic inhibitor where the gain of copy number of MYC is greater than or equal to five copies and the gain in copy number of HSF1 is greater than or equal to five copies in at least 5% of the cancer cells in the subject sample.

A thirty-fifth embodiment is a method for identifying potential therapeutic targets for efficacy in cancer cells by performing the steps of obtaining at least one MYC-HSF1 coamplified cell line and at least one MYC-HSF1 non-coamplified cell line; treating the coamplified cell line and the non-coamplified cell line with an agent having a known therapeutic target; obtaining the average relative cell viability for the coamplified cell line and the average relative cell viability for the non-coamplified cell line; and subtracting the average relative cell viability of the non-coamplified cell line from the average relative cell viability of the coamplified cell line to arrive at a positive or negative value, wherein the positive value is reflective of a greater response in the coamplified cell line.

A thirty-sixth embodiment is a method for identifying potential therapeutic compound having efficacy in cancer cells by performing the steps of obtaining at least one MYC-HSF1 coamplified cell line and at least one MYC-HSF1 non-coamplified cell line; treating the coamplified cell line and the non-coamplified cell line with a therapeutic agent; obtaining the average relative cell viability for the coamplified cell line and the average relative cell viability for the non-coamplified cell line; and subtracting the average relative cell viability of the non-coamplified cell line from the average relative cell viability of the coamplified cell line to arrive at a positive or negative value, wherein the positive value is reflective efficacy of the therapeutic compound against the coamplified cell line.

### BRIEF DESCRIPTION OF THE FIGURES

The accompanying drawings are included to provide a further understanding of the disclosure and are incorporated in and constitute a part of this specification, illustrate embodiments, and together with the description serve to explain the principles of the disclosure.

FIG. 1A shows a bar graph depicting copy number variation (CNV) for MYC and HSF1 across cancer types in The Cancer Genome Atlas (TCGA) cohorts. Data was analyzed via cBioPortal (www.cbioportal.org).

FIG. 1B is a table showing Chi-Square Analysis of the copy number variation of MYC and HSF1. Venn diagram drawn to scale to represent all ovarian cancer tumors that had MYC and HSF1 gene amplifications.

FIG. 1C is a heat map depicting the copy number variation of each chromosome in all TCGA ovarian cancer cohort (TCGA-OV) patients. Chromosome 8, where MYC and HSF1 are located, is indicated by the box.

FIG. 2A and FIG. 2B are graphs depicting MYC and HSF1 activity estimated in the TCGA-OV cohort using published gene signatures for each transcription factor. Pearson correlation was performed to assess the relationship between MYC and HSF1 activity.

FIG. 2C is an immunoblot depicting the result in each ovarian cancer cell line (indicated along the top) when subjected to immunoblotting with the indicated antibody (indicated to the right). Cells with MYC and HSF1 gene amplification are labelled (+) underneath.

FIG. 2D is a graph depicting quantification of the immunoblotting shown in FIG. 2C and the Pearson correlation between MYC and active HSF1 (pS326).

FIG. 2E shows MYC-amplified TCGA-OV patients were further separated by high and low HSF1 activity using a published HSF1 gene signature. Log Rank test was performed for statistical significance.

FIG. 2F represents OVCAR8 cells transiently transfected with control, MYC, or HSF1 siRNA, and total protein subjected to immunoblotting with indicated antibodies.

FIG. 2G represents OVCAR8 cells subjected to a luciferase reporter assay using a reporter with multiple heatshock elements (HSE) as a readout for HSF1 activity with control, MYC, or HSF1 siRNA.

FIG. 2H represents FLAG-tagged HSF1 expressed in OVCAR8 cells and total protein subjected to coimmunoprecipitation with FLAG antibodies and immunoblotting with indicated antibodies.

FIG. 3A is a diagram of the relationship between PLK1 and MYC/HSF1 indicating PLK1 can directly regulate MYC and HSF1 through phosphorylation but also indirectly by regulating the PI3K-AKTpathway.

FIGS. 3B-3E represent a cohort (n=100) of ovarian cancer patient tumor specimens which were subjected to immunohistochemistry (IHC) with indicated antibodies (indicated along the top). The High/Low designation is in reference to the amount of MYC/HSF1 in the sample case tumor. Low group indicates a case where the amount of both MYC and HSF1 are low and the High group indicates a case where the amount of both MYC and HSF1 are both high (FIG. 3B). QuPath-quantified results were subjected to Pearson correlation between HSF1 and MYC (FIG. 3C), p-PLK1 and MYC (FIG. 3D), and p-PLK1 and HSF1 (FIG. 3E).

FIGS. 4A-4C represent the assessment of the IC50 value for volasertib in the indicated ovarian cancer cell lines with (+AMP) or without (−AMP) MYC-HSF1 dual amplification. The average data is provided in table format (FIG. 4A), in graphical format as log BI-6727 (μM) for the −AMP and +AMP groups (FIG. 4B) and in graphical format of % viability as compared to log BI-6727 (μM) for each of the tested cancer cell lines (FIG. 4C).

FIG. 4D shows immunoblots of OVCAR8 cells (a +AMP cell line) treated with volasertib (1 nM) for the indicated time points and immunoblotting total protein with the indicated antibodies.

FIG. 4E is a bar graph representing OVCAR8 cells subjected to HSE luciferase assay with treatment of volasertib (1 nM) for the indicated times.

FIG. 5A depicts bar graphs showing colonogenic growth observed for OVCAR8, OVCAR4 and SNU119 cell lines subjected to the indicated dose of volasertib (BI-6727). Control cells were subjected to 1% DMSO instead of volasertib. Colonies were grown for 7 days prior to staining with crystal violet and quantification was completed with FIJI software.

FIG. 5B and FIG. 5C represent OVCAR8 (MYC-HSF1 co-amplified) and CAOV3 (non-amplified) cells subjected to tumor spheroid growth in the presence or absence of volasertib at the indicated doses. Spheroids were quantified by manual counting. The impact volasertib on spheroid growth in the two different cell lines is shown photographically (FIG. 5B) and graphically (FIG. 5C).

FIG. 5D represents OVCAR8 cells (5e5) grown in the flank of nude mice until a volume between 50-100 mm³ were achieved. Mice were then randomized to receive either vehicle, 15 mg/kg, or 20 mg/kg volasertib twice per week. Tumor volume was measured with calipers (FIG. 5D) and body weight was measured (FIG. 5E) throughout the study.

FIG. 6A is a graph illustrating difference in cell viability relative to control (1% DMSO) observed in drug screen with an epigenetic inhibitor library on ovarian cancer cells with (OVCAR4, OVCAR8) or without (OVSAHO, CAOV3) dual amplification of MYC and HSF1.

FIG. 6B is a bar graph depicting mRNA relative fold change in OVCAR8 cells treated with entinostat at the indicated doses for 24 hours. Total RNA from the cells was subjected to RT-qPCR for the indicated transcripts.

FIG. 6C and FIG. 6D are immunoblots of OVCAR8 cells treated with entinostat at the indicated doses for 24 hours. Total protein from the cells was subjected to immunoblotting with the indicated antibodies.

### DETAILED DESCRIPTION

Ovarian cancer is a deadly female cancer that is frequently diagnosed at advanced stages, leading to poor patient outcomes. MYC is a frequent oncogenic driver across many tumor types, including ovarian cancer. The MYC gene (c-MYC) encodes a nuclear phosphoprotein that plays a role in cell cycle progression, apoptosis and cellular transformation in normal human cells. See, e.g., Dang, Cell. 2012 Mar. 30; 149(1): 22-35. However, MYC is also frequently amplified in cancer cells and has long been known as an oncogene that promotes several mechanisms that induce oncogenesis and promote cancer progression. MYC was found to be amplified in 45% of ovarian cancer patients and MYC expression is also a reported prognostic marker for response to chemotherapy in patients with ovarian cancer.

Interestingly, MYC was frequently co-amplified with another transcription factor, heat shock factor 1 (HSF1). HSF1 is a transcription factor that was originally discovered as the master regulator of the heat shock response. This role of HSF1 includes the transcriptional upregulation of chaperone heat shock proteins in response to cellular stressors. HSF1 has also been shows to impact carcinogenesis. See, e.g., Dai et al., Cell 2007 Sept; 130(6):1005-1018. Beyond this known physiological role, HSF1 is overexpressed and/or hyperactivated in many tumor types, including breast cancer. In breast cancer, high HSF1 activity has previously been associated with poor patient outcomes. We have found that 35% of ovarian cancer patients also had dual amplification of the genes for these two transcription factors-MYC and HSF1. Non-amplified gene copy numbers of MYC and HSF1 can range from 0-2 copies of each gene, whereas dual amplification of MYC and HSF can involve gene copies of 3, 4, 5, 6, 7, 8, or greater for each gene. See, e.g., Vita & Henriksson, Semin Cancer Biol. 2006 August; 16(4):318-30 and Tansey, New Journal of Science, 2014 February;2014:757534. Copy number can be determined according to a variety of methods available to one of skill in the art, including, e.g., fluorescence in situ hybridization, DNA, microarrays, comparative genomic hybridization, polymerase chain reaction, next-generation sequencing, southern blot analysis, immunohistochemistry, or a combination thereof. See, e.g., Carter, Nat Genet. 2007 July; 39(7 Suppl): S16-S21, Shayeb et al., JCO Precision Oncology 2023 Sept;7(7); and Nakamura et al., Med Oncol. 2021 Mar. 12;38(4):36.

Previous reports have also suggested a functional interaction between MYC and HSF1, including loss of HSF1 prevents hepatocellularcarcinogenesis (HCC) in a MYC-driven mouse model of HCC. Cigliano, et al., Oncotarget 2017; 8:90638-50. While the molecular interactions between MYC and HSF1 were not clear from this study, it does indicate MYC being dependent on HSF1 to act as an oncogenic driver. Some further indications of the molecular interactions between MYC and HSF1 were detailed in a recent report that suggests HSF1 potentiates MYC transcriptional activity independent of HSF1-driven expression of chaperones. Xu, et al., bioRxiv 2022:2022.02.22.481519. This distinction is crucial because a major regulation point for MYC is protein stability with a very short half-life that could be impacted by chaperone-mediated MYC stability. Interestingly, report by Xu, et al. indicated that transcription-deficient HSF1 can still potentiate MYC transcriptional activity indicating HSF1 interaction with MYC is driving the transcription of MYC. While this recent report was not studied in the context of cancer, the current results support that MYC and HSF1 also form a protein complex in ovarian cancer cells. Distinct from the non-cancer setting, the Xu et al. results indicate HSF1 may also be critical for maintaining MYC expression at the transcript level. We found that a loss of HSF 1significantly reduced MYC levels in dual-amplified ovarian cancer cells while loss of MYC did not impact HSF1 expression or function. Additionally, these two transcription factors were observed to form a complex, suggesting they are functionally cooperating.

Grammatical variations of “administer,” “administration,” and “administering” to a subject include any route of introducing or delivering to a subject an agent. Administration can be carried out by any suitable route, including oral, topical, intraveneous, subcutaneous, transcutaneous, transdermal, intramuscular, intra joint, parenteral, intra-arteriole, intradermal, intraventricular, intracranial, intraperitoneal, intralesional, intranasal, rectal, vaginal, by implanted reservoir, and the like.

The term “amplification” means an increase in copy number of a gene. For purposes of the present disclosure, the terms “amplification,” “copy number gain” and “gain in copy number” are used interchangeably as both are understood to represent an increase in copy number of a gene above the number present cells having normal cellular function. One of skill in the art would appreciate the term amplification reflects an increase in copy number of the gene above that of gain in copy number. For example, copy number gain of a gene may represent a mean copy number of 3-4 copies in ≥5% of cells while amplification of that same gene would be understood to mean the presence of ≥5 copies per cell in ≥5% of analyzed cells. Software programs, such as cBioPortal (www.cbioportal.org), may be utilized to determine the magnitude of increase in copy number of a gene sequence in a sample. Furthermore, it is understood by those of skill in the art that the term “amplification” is distinct from the term “overexpression,” as the term “overexpression” refers to an increase in mRNA or protein levels. While overexpression can be present where there is an increase in copy number of a gene, overexpression of mRNA or protein may also occur in a cell when there is no increase in gene copy number.

The term “inhibitor” means a molecule that impedes or decreases a biological action. For the purpose of the present disclosure, an inhibitor generally has a specific target in the cell that it inhibits but inhibition of that specific target will have indirect effects on other biological molecules or processes that are regulated by the specific target. This inhibition may happen directly or indirectly. By way of example, and in no way limiting the term inhibitor as used herein, the inhibitor of PLK 1 is understood to directly inhibit the protein function of PLK1 and then indirectly inhibit the activity and protein stability of MYC and HSF1.

“Therapeutically effective amount” or “therapeutically effective dose” of a composition (e.g. a composition comprising an agent) refers to an amount that is effective to achieve a desired therapeutic result (e.g., reducing the size of a tumor or reducing cancer cell proliferation). Therapeutically effective amounts will typically depend upon the IC50 and safety profile of the specific agent being administered.

### Methods Using Co-Amplification of MYC and HSF1 as Biomarkers for Response to Cancer Therapeutics

In some aspects, provided herein are methods of determining enhanced sensitivity of cells, e.g., a biological sample having a population of dual amplified MYC-HSF1 gene copy number, to a therapeutic inhibitor, such as an HDAC inhibitor. To determine if MYC and HSF1 co-amplification could serve as a biomarker for response to cancer therapeutics, Applicant evaluated the impact of polo-like kinase 1 (PLK1) in co-amplified and non co-amplified ovarian cancer cells. PLK1 has been a viable therapeutic target in cancer for many years. Active PLK1 was positively correlated with both MYC and HSF1 levels in ovarian cancer patient tumor specimens. The kinase PLK1 has been shown to directly phosphorylate both MYC and HSF1 to enhance their activity and protein stabilization. PLK1 is a serine/threonine kinase most known for its functions in regulating several aspects of the cell cycle. PLK1 has been shown to be overexpressed in a wide range of human cancers, including ovarian cancer, and had associations with poor patient outcomes. Aside from phosphorylating both MYC and HSF1, PLK1 can also directly phosphorylate and deactivate the phosphatase PTEN thereby enhancing activity of PI3K-AKT1 signaling.

The PLK1-specific inhibitor volasertib was found to have a 200-fold enhanced efficacy in MYC-HSF1 dual amplified ovarian cancer cells compared to cells without this dual amplification. Volasertib (BI-6727) is a selective PLK1 inhibitor that has shown promise as an effective cancer therapy in early phase clinical trials (Rudolph et al. Clin Cancer Res 2009; 15:3094-102; Pujade-Lauraine, et al. J Clin Oncol 2016; 34:706-13; Xie, et al. Am J Cancer Res 2015; 5:3548-59). Additionally, these clinical trials also indicated volasertib had favorable toxicity and safety for use in humans leading to advanced clinical trials in multiple tumor types. Interestingly, there has been one trial testing volasertib in ovarian cancer patients where single agent volasertib showed a 30%24-week disease control rate, 13% of patients showed partial responses, and 11% of patients achieved progression-free survival for more than one year compared to none in patients receiving cytotoxic chemotherapy (Pujade-Lauraine et al. J Clin Oncol 2016; 34:706-13). Therefore, single-agent volasertib showed potential for antitumor activity in this trial.

In this disclosure, we show that treatment with volasertib reduced protein levels of both MYC and HSF1 consistent with the effect of PLK1 phosphorylation on MYC and HSF1 protein stability. Volasertib was highly effective at reducing growth of MYC-HSF1 dual amplified ovarian cancer cells in clonogenic growth assays, tumor spheroid assays, and in vivo tumor growth. These data identify a novel interaction between MYC and HSF1 in ovarian cancer and identify MYC-HSF1 dual amplification as a biomarker for therapeutic response to inhibition of PLK1.

The current results indicate that patients that have MYC-HSF1 dual amplification may have an enhanced response to PLK1 inhibitor therapeutics like volasertib and the ovarian cancer patients that responded in this study could have enhanced MYC-HSF1 function leading to their positive response but this was not determined. Together, these data indicate MYC and HSF1 dual amplification appears to be significant driver for more than one-third of ovarian cancer patients and this genetic alteration could serve as a biomarker for treatment with PLK1 targeting agents. The frequency of sequencing ovarian cancer patient tumors following surgical resection also makes it feasible to identify patients with these gene amplifications for determination of whether a patient would benefit from PLK1 inhibitors such as volasertib.

### Methods of Using Co-Amplification of MYC and HSF1 as Biomarkers Screening Potential Cancer Therapeutics

In some aspects, provided herein are methods of screening therapeutic agents, such as epigenetic inhibitors, against a biological sample comprising a population of cells having dual amplification of MYC and HSF1 genes. In one example, a cell having greater than or equal to 3 gene copies of MYC and greater than or equal to 3 gene copies HSF1 is considered to have dual amplification of MYC and HSF1 genes. Applicant demonstrated that MYC and HSF1 dual amplification could serve as a biomarker for treatment of ovarian cancer cells with PLK1 targeting agents, we evaluated whether MYC and HSF1 dual amplification could serve as a biomarker for other potential therapeutics for these cancer cells as well. We evaluated 415 unique epigenetic inhibitors (potential cancer therapeutics) on two cell lines with MYC-HSF1 amplification (OVCAR8, OVCAR4) and two cell lines without these genes amplified (OVSAHO, CAOV3). Results of this screen indicated 45 of the top 54 hits were compounds that targeted histone deacetylases (HDACs). We tested an HDAC inhibitor, entinostat, against co-amplified and non co-amplified ovarian cancer cells to evaluate whether the screening using MYC and HSF1 dual amplification biomarker identified a potential efficacious therapeutic against the co-amplified cells. Treatment using an HDAC inhibitor resulted in reduced protein levels of both MYC and HSF1 in the co-amplified cells.

Epigenetic inhibitors are involved in alterations at the level of DNA, e.g., DNA methylation and histone deacetylation. See, e.g., Verma & Banerjee, Methods Mol Biol. 2015:1238:469-85. Exemplary epigenetic inhibitors include DNA methyltransferase inhibitors, histone deacetylase inhibitors, histone methyltransferase inhibitors, bromodomain and extra-terminal domain inhibitors, and lysine-specific demethylase 1 (LSD-1) inhibitors.

### Method of Treatment

In some aspects, provided herein are methods of treating cancer in a subject, such as where a biological sample of cancer cells obtained from the subject has greater than or equal to three gene copies of MYC and greater than or equal to three gene copies of HSF1. In some examples, the biological sample may include a population of cells having a gene copy number of MYC that is 3, 4, 5, 6, 7, 8 or greater and a gene copy number of HSF1 that is 3, 4, 5, 6, 7, 8 or greater. In some embodiments, the biological sample comprises prostate cancer cells, bladder cancer cells, breast cancer cells, ovarian cancer cells, colorectal cancer cells, lung cancer cells, or esophageal cancer cells.

In some embodiments, disclosed methods involve administering an epigenetic regulator to the subject. In some embodiments, disclosed methods involve administering an HDAC inhibitor to the subject. HDAC inhibitors are described, e.g., by Kim & Bae, Am J Transl Res. 2011 Jan. 1; 3(2): 166-179 and Eckschlager et al., Int J Mol Sci. 2017 July; 18(7): 1414. Exemplary HDAC inhibitors include entinostat, vorinostat, romidepsin, panobinostat, belinostat, analogs thereof, and pharmaceutical salts thereof. In some embodiments, disclosed methods involve administering a PLK-1 inhibitor to the subject. PLK-1 inhibitors are described, e.g., by Chiappa et al., Front Oncol. 2022; 12: 903016. Exemplary PLK-1 inhibitors include volasertib, BI2536, BI6727, NMS-1286937, GSK461364, analogs thereof, and pharmaceutical salts thereof. In some embodiments, disclosed methods involve co-administering an HDAC inhibitor and a PLK-1 inhibitor to the subject. In some embodiments, the subject has ovarian cancer, prostate cancer, bladder cancer, breast cancer, ovarian cancer, colorectal cancer, lung cancer, or esophageal cancer. In some embodiments, the subject is human.

Efficacy of disclosed treatment methods can be determined according to knowledge available to one of skill in the art. In one example, direct tumor response to treatment may be determined by imaging or detection of biomarkers. Determining inhibition of growth or spread may also be implicated in determining treatment efficacy. In other examples, reduction in pain and other symptoms and increased quality of life may indicate treatment efficacy. See also, e.g., Eckschlager et al., Int J Mol Sci. 2017 July; 18(7): 1414; Orr & Edwards, Hematol Oncol Clin North Am. 2018 December; 32(6):943-964; and Eisenhauer, Ann Oncol. 2017 Nov. 1; 28(suppl_8):viii61-viii65.

### EXAMPLES

### Example 1: Analysis of Frequency of Copy Number Gain of both MYC and HSF1 in Ovarian Cancers

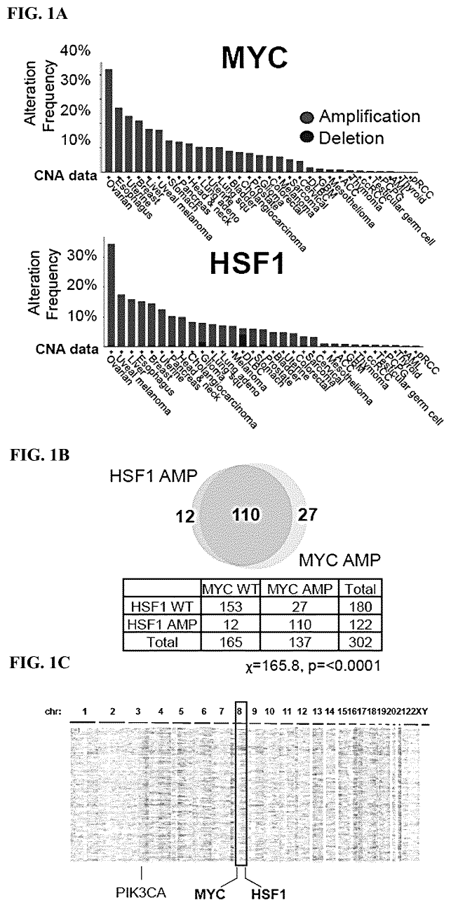

MYC has previously been reported to be overexpressed in >60% of ovarian tumors (10-17). Analysis of the ovarian The Cancer Genome Atlas (TCGA) cohorts indicates the MYC gene is amplified in 45% of cases (FIG. 1A-FIG. 1C), which is likely a significant contributor to MYC overexpression.

The HSF1gene was also highly amplified in ovarian tumors (40% of cases) (FIG. 1A-FIG. 1C). Ovarian cancer has the highest frequency for gene amplification for both MYC and HSF1 (FIG. 1A). The coamplification of both MYC and HSF1 was a significant co-occurrence (p<0.0001) with 36% of all patients having the dual amplification of these two genes (FIG. 1B). While these two genes are both on the long arm of chromosome 8 (FIG. 1C), these two genes can be separately amplified (FIG. 1B), suggesting they are on separate amplicons. However, the frequency for which they are co-amplified suggests the tumor cell obtains a growth or survival advantage with dual amplification.

Gene amplification status in all TCGA cohorts was determined using called amplification status from publicly-available TCGA in cBioPortal. RNA-sequencing of the TCGA ovarian cancer cohort (TCGA-OV) were downloaded in RPKM (reader per kilobase of exon per million reads mapped) for analyses. MYC activity was assessed using a published gene signature (Gatza et al., PNAS US 2010; 107:6994-9). Similarly, HSF1 activity was assessed using our recently-identified HSF1 activity signature (HAS) (Jacobs et al. bioRxiv 2022:2022.05.12.491688). Survival analysis was performed using Kaplan-Meier plots with Log Rank test for significance.

A person of skill in the art would appreciate that in addition to cBioPortal, the gene amplification analysis could also be performed using the raw data that can be accessed from the TCGA Data Portal (https://portal.gdc.cancer.gov/). Additionally, similar though not identical raw ovarian cancer data is also available through the NCBI Gene Expression Omnibus (GEO).

### Example 2: Analysis of Functional Linkage of MYC and HSF1 in Ovarian Cancer Cells

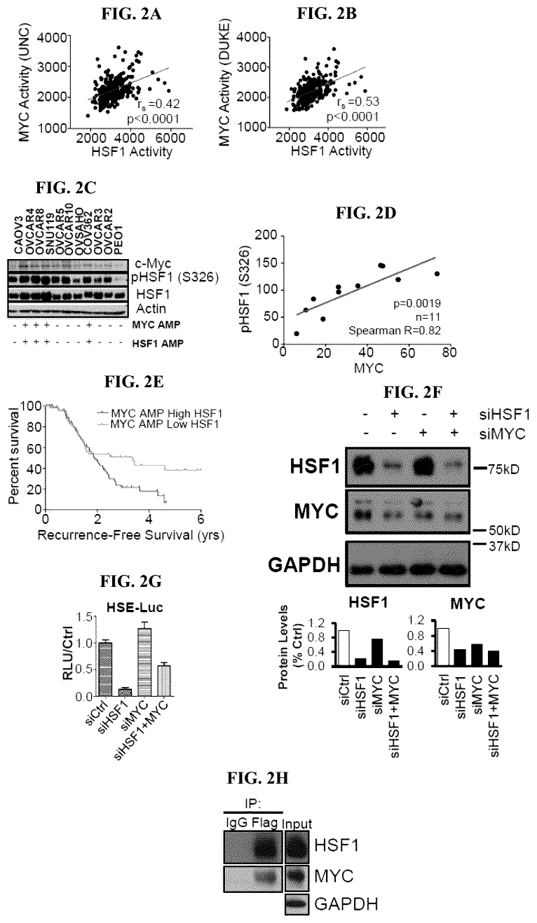

Because HSF1 was previously indicated as critical for the ability of MYC to bind and transactivate MYC target genes, gene signatures were used to assess MYC and HSF1 activity to determine if MYC and HSF1 are functionally linked in ovarian cancer. Pearson correlation was performed to assess the relationship between the MYC and HSF1 activity and these results indicated a strong and significant positive association (FIG. 2A and FIG. 2B), suggesting these two transcription factors may be cooperating.

Assessment of ovarian cancer cell lines identified four cell lines with MYC and HSF1 dual amplification (FIG. 2C) and that the active form of HSF1 (pS326) is highly correlated with MYC levels (FIG. 2D).

Further supporting the cooperation of HSF1 and MYC as drivers for ovarian cancer is that tumors with high HSF1activity and MYC amplification have worse recurrence-free survival compared to those with low HSF1 activity (FIG. 2E). A prior report indicated HSF1 was likely acting as a co-factor for MYC but did not appear to regulate expression of MYC (Xu et al. bioRxiv 2022:2022.02.22.481519). However, our analysis shows that loss of HSF1 resulted in decreased levels of MYC in ovarian cancer cells (FIG. 2F). Thus, the amount of MYC expression is dependent upon HSF1. Xu et al. did not find that the amount of MYC expression was dependent on HSF1, but that study was performed in non-cancer cells. In contrast, loss of MYC did not affect HSF1 levels or HSF1 activity (FIG. 2F and FIG. 2G), suggesting HSF1 may be important for MYC expression and function but MYC does not appear to regulate HSF1 function.

FLAG-tagged HSF1 was able to pull down MYC-indicating these two transcription factors can form a complex in ovarian cancer cells (FIG. 2H), which is consistent with the previous report showing these transcription factors form a complex in non-cancer cells.

### Example 3: MYC and HSF1 are Associated with PLK1

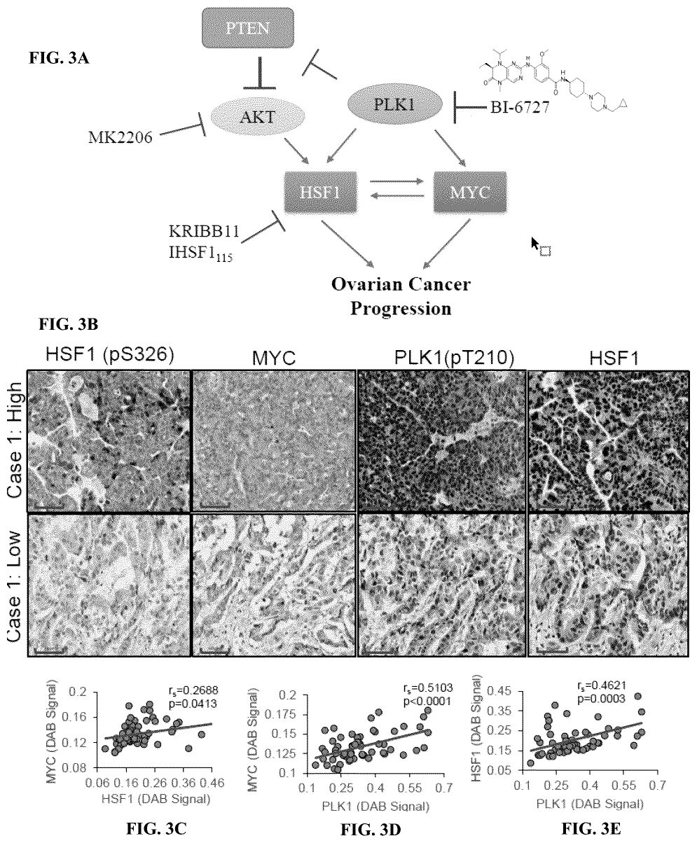

To identify possible therapeutic approaches that would benefit ovarian tumors with MYC and HSF1 dual amplification, polo-like kinase 1 (PLK1) was identified as a kinase that can phosphorylate and regulate both MYC and HSF1. Aside from phosphorylating both MYC and HSF1, PLK1 can also directly phosphorylate and deactivate the phosphatase PTEN thereby enhancing activity of PI3K-AKT1 signaling. Our work indicated that AKT1 is an activator of HSF1 and plays a key role in the early stages of metastasis. Therefore, PLK1 can both directly and indirectly enhance activity of MYC and HSF1 (FIG. 3A). In a cohort of 100 ovarian patient tumor specimens, we found a positive association between MYC and HSF1 levels as well as a positive association with active PLK1 (T210) with MYC and HSF1 (FIGS. 3B-3E).

### Example 4: PLK1 Inhibition is More Effective with MYC and HSF1 Dual Amplification

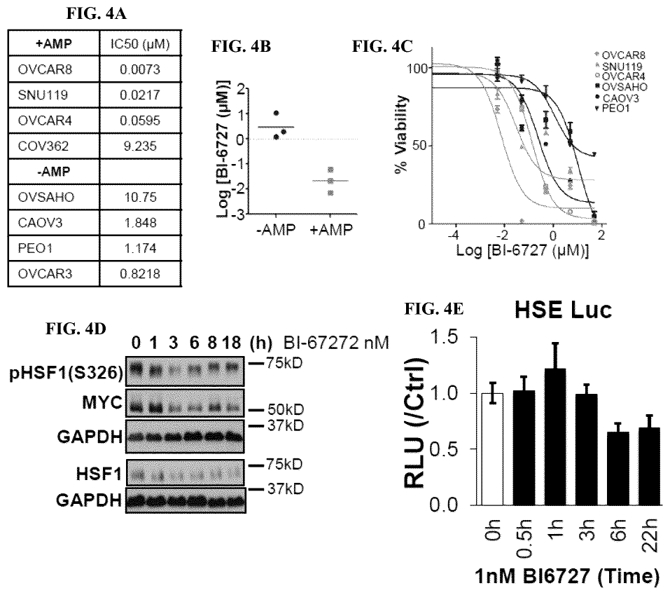

PLK1 is an active therapeutic target with several compounds targeting this kinase in clinical trials. One of the therapeutic agents is volasertib (BI-6727), which is a selective PLK1 inhibitor that has shown promise as an effective cancer therapy in early phase clinical trials (Rudolph et al. Clin Cancer Res 2009; 15:3094-102; Pujade-Lauraine et al. J Clin Oncol 2016; 34:706-13; Xie et al. Am J Cancer Res 2015; 5:3548-59). These clinical trials of volasertib also indicated that the therapeutic agent had favorable toxicity and safety for use in humans leading to advanced clinical trials in multiple tumor types. To assess whether a PLK1 inhibitor has specificity for MYC-HSF 1 dual-amplified ovarian cancer cells, the IC50 for volasertib was assessed in cell lines with dual amplification (OVCAR8, SNU119, OVCAR4, and COV362) and in cell lines without dual amplification (OVSAHO, CAOV3, PEO1, OVCAR3). Intriguingly, the IC50 for volasertib in MYC-HSF1 dual amplified cells was 33 nM whereas the IC50 in non-amplified cells was 7.8 μM, for a >200-fold difference (FIGS. 4A-4C). These results demonstrated volasertib efficacy is greater in MYC-HSF1 dual amplified ovarian cancer cells.

This data demonstrating volasertib's improved efficacy in MYC-HSF1 dual amplified ovarian cancer cells, also demonstrates the underestimation of volasertib's efficacy in prior clinical trials. A person skilled in the art would understand that based upon the present disclosure, in a clinical trial 100 random patients, 35 of those patients would have MYC-HSF1 dual amplification. Therefore, if only patients having MYC-HSF1 dual amplification are going to respond to volasertiv, then only 35 of 100 patients in the study would have an effective therapeutic response. Because the impact of MYC-HSF1 dual amplification was unknown prior to this disclosure, the patient population for volasertib could not be segregated based upon amplification, which is reflected in the responsiveness observed in the prior clinical trials of volasertib. Thus, designing a clinical trial in accordance with the present disclosure, where 100% of the patients have MYC-HSF1 dual amplification cancer cells, is anticipated to provide a more accurate reflection of the percentage of patients that would benefit from the drug being tested.

Considering PLK1 phosphorylation previously indicated affects the protein stability of MYC and HSF1, it was tested whether volasertib affects MYC or HSF1 protein levels. A time course study was performed where samples were treated with volasertib for time ranges of 0 to 18 hours. This time course of volasertib exposure indicated a loss of MYC and HSF1 protein after 3 hours of volasertib exposure, suggesting the effectiveness of volasertib is likely related to the regulation of MYC and HSF1 (FIG. 4D). Similarly, the effect of volasertib was tested on the activity of HSF 1 using an HSE luciferase reporter. Results indicated that volasertib with increasing time of treatment led to increasing inhibition of HSF1 activity (FIG. 4E).

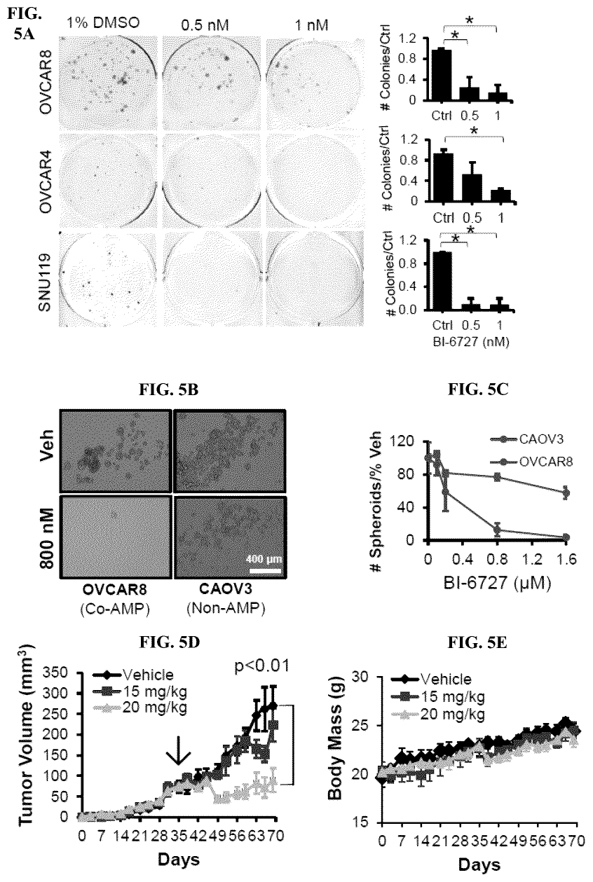

The effectiveness of volasertib on MYC-HSF1 dual amplified cancer cells was also analyzed in several cell growth models. First, volasertib effectiveness was tested in clonogenic (colony forming) growth assays, which indicated strong inhibition of clonogenic growth in MYC-HSF1 dual amplified cells (FIG. 5A). Cell viability assays were performed with Cell Titer Blue (Promega) as we previously described (Carpenter et al. Oncotarget 2017; 8:73947-63). Clonogenic growth assays were performed by seeding <1000 cells into 6-well plates and staining with crystal violet after 7 days of growth. Colonies were quantified using FIJI.

Additionally, volasertib was observed to inhibit spheroid growth of MYC-HSF1 dual amplified ovarian cancer cells but not cells without these genes amplified (FIG. 5B and FIG. 5C). Ovarian cancer cells (1000-2000) were seeded into 24-well ultra-low attachment plates (Corning) and grown in serum-free spheroid media as we previously described (Wang et al., Mol Cancer Ther 2021). Spheroids were grown in the presence or absence of volasertib for 7 days. Spheroids were quantified by manual counting.

Lastly, we evaluated the efficacy of the PLK 1 inhibitor, volasertib, on tumor growth in vivo. OVCAR8 cells (5e5) were injected into the flank of nude mice and tumors were allowed to develop until 50-100 mm³. Mice then received either vehicle (PBS) or 15 or 20 mg/kg volasertib twice per week for five weeks. Body weight and tumor volume were measured twice per week. Volasertib was seen to strongly regress tumor growth in vivo of MYC-HSF1 dual amplified ovarian cancer cells (FIG. 5D). Mice tolerated volasertib treatment well as indicated by no change in body weight with any dose of volasertib (FIG. 5E).

### Example 5: Using Dual Amplification of MYC-HSF1 to for Cancer Drug Screening

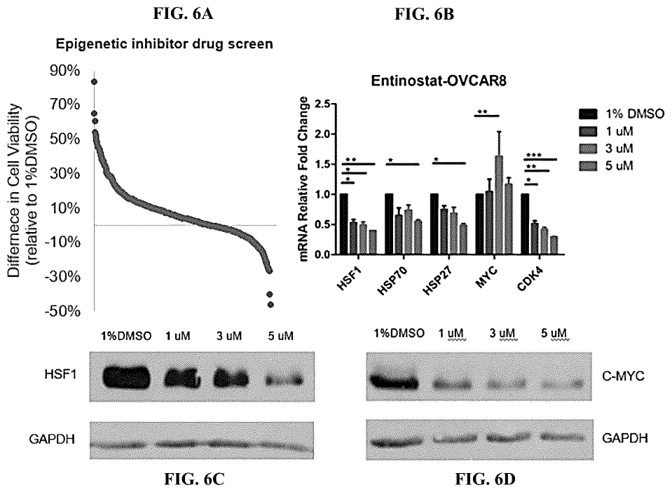

Drug screen was performed on two cell lines with MYC-HSF1 amplification (OVCAR8, OVCAR4) and two cell lines without these genes amplified (OVSAHO, CAOV3). As shown in Table 1, cell lines were treated with the indicated drug for 48 hrs and cell viability was measured using Cell Titer Blue kit (Promega). Epigenetic drug libraries were purchased from Cayman Chemical (Catalog #: 11076) and APExBIO (Catalog #: L1029). Together, these two libraries used tested 415 unique epigenetic inhibitors (potential cancer therapeutics). Results were analyzed to indicate the relative cell viability of cells after treatment compared to cells receiving vehicle (1% DMSO). Relative cell viability for cells positive or negative for MYC-HSF1 amplification were averaged across these groups of cell lines. To determine specificity of response for cells with or without the biomarker of MYC-HSF1 dual amplification, average relative cell viability for cells negative for amplification was subtracted by average relative cell viability for cells positive for amplification. After this subtraction, compounds with a positive value had greater response in cells positive for MYC-HSF1dual amplification and compounds with a negative value had a greater response in cells negative for MYC-HSF1 dual amplification. This data is also reflected graphically in FIG. 6A.

TABLE 1
Results of Epigenetic Inhibitor Drug Screen

| | | Non Amp - AMP | |
|---|---|---|---|
| | Drug Name | (POC) 10 uM | Target |
| 1 | CXD101 | 83.34562498 | HDAC |
| 2 | MS-275 (entinostat) | 65.05896708 | HDAC |
| 3 | Pyroxamide | 60.34156632 | HDAC |
| 4 | UF010 | 54.18348853 | HDAC |
| 5 | Entinostat (MS-275, SNDX-275) | 52.31389866 | HDAC |
| 6 | RGFP109 | 50.51948869 | HDAC |
| 7 | Mocetinostat | 49.31495628 | HDAC |
| 8 | CAY10398 | 48.60751409 | HDAC |
| 9 | Trichostatin A | 47.3402202 | HDAC |
| 10 | Apicidin | 45.97401547 | HDAC |
| 11 | UNC 0646 | 45.25386209 | HMT |
| 12 | Scriptaid | 45.23909414 | HDAC |
| 13 | LMK 235 | 44.95847382 | HDAC |
| 14 | SAHA | 44.78525583 | HDAC |
| 15 | CAY10603 | 43.88584772 | HDAC |
| 16 | LAQ824 | 43.85941779 | HDAC |
| 17 | PCI 24781 | 42.57451033 | HDAC |
| 18 | SB939 | 42.19157727 | HDAC |
| 19 | Panobinostat | 41.45677753 | HDAC |
| 20 | ITF 2357 | 40.35202149 | HDAC |
| 21 | Hesperadin | 39.85195802 | Aurora Kinase |
| 22 | GSK591 | 38.4203614 | HMT |
| 23 | JNJ-26481585 | 38.34489078 | HDAC |
| 24 | CBHA | 37.52961455 | HDAC |
| 25 | Vorinostat (SAHA, MK0683) | 37.32478765 | HDAC |
| 26 | Mocetinostat (MGCD0103, MG0103) | 36.87948315 | HDAC |
| 27 | Resminostat (hydrochloride) | 36.33104033 | HDAC |
| 28 | ACY-241 | 35.16729223 | HDAC |
| 29 | LMK 235 | 34.7705648 | HDAC |
| 30 | UNC 0631 | 34.73112651 | HMT |
| 31 | Suberohydroxamic Acid | 34.24500501 | HDAC |
| 32 | Chidamide | 33.84685941 | HDAC |
| 33 | EED226 | 32.63892329 | HMT |
| 34 | Rocilinostat (ACY-1215) | 32.22512488 | HDAC |
| 35 | BML-210 | 30.70283165 | HDAC |
| 36 | Scriptaid | 29.99140419 | HDAC |
| 37 | PAOA | 29.57403161 | HDAC |
| 38 | 4-iodo-SAHA | 29.49846407 | HDAC |
| 39 | CAY10603 | 29.04757398 | HDAC |
| 40 | LAQ824 (NVP-LAQ824, Dacinostat) | 28.82395079 | HDAC |
| 41 | OG-L002 | 28.78535559 | HDM |
| 42 | PCI-24781 (CRA-024781) | 28.70045269 | HDAC |
| 43 | Gemcitabine | 28.61260894 | Nucleoside |
| 44 | 3-Deazaneplanocin A | 28.52089584 | HMT |
| 45 | Trichostatin A (TSA) | 28.27719184 | HDAC |
| 46 | CUDC-101 | 28.2551892 | HDAC |
| 47 | NSC 3852 | 27.71372456 | HDAC |
| 48 | NCH-51 | 27.52298211 | HDAC |
| 49 | Panobinostat (LBH589) | 27.26081901 | HDAC |
| 50 | JNJ-26481585 | 26.24768711 | HDAC |
| 51 | RSC-133 | 26.08018446 | DNMT, HDAC |
| 52 | BG45 | 25.7370442 | HDAC |
| 53 | CI-994 | 25.1847451 | HDAC |
| 54 | Oxamflatin | 24.32789919 | HDAC |
| 55 | AGK 2 | 24.16668027 | Sirtuin |
| 56 | Nexturastat A | 23.67354146 | HDAC |
| 57 | 2-Methoxyestradiol (2-MeOE2) | 23.41183277 | Apoptosis Inducers |
| 58 | Pracinostat (SB939) | 22.93163457 | HDAC |
| 59 | BVT 948 | 22.8658182 | HMT |
| 60 | M344 | 22.64569972 | HDAC |
| 61 | UNC0379 | 21.91678926 | HMT |
| 62 | UF 010 | 21.85855626 | HDAC |
| 63 | CID 2011756 | 21.7907516 | Protein Ser/Thr Phosphatases |
| 64 | I-BET151 | 21.71870942 | BET |
| 65 | Bromosporine | 21.42563134 | Bromodomain |
| 66 | (+)-JQ1 | 20.81660049 | BET |
| 67 | Tenovin-1 | 20.59560319 | SIRT |
| 68 | I-CBP112 (hydrochloride) | 20.47974479 | HAT |
| 69 | Picolinamide | 20.34907239 | PARP |
| 70 | 5-Azacytidine | 20.04848555 | DNA Methyltransferase |
| 71 | AGK2 | 19.95617683 | SIRT |
| 72 | AMI-1 | 19.64446093 | HMT |
| 73 | Tubastatin A HCl | 19.35048059 | HDAC |
| 74 | (R)-PFI-2 (hydrochloride) | 19.34785641 | HMT |
| 75 | BMS-345541 (free base) | 19.27411751 | HDAC |
| 76 | GSK2801 | 19.16768615 | NoRC |
| 77 | CAY10683 | 18.97734703 | HDAC |
| 78 | SIRT 1/2 Inhibitor IV | 18.9487227 | SIRT |
| 79 | AMI-1 | 18.33684544 | PRMT |
| 80 | HPOB | 18.14467164 | HDAC |
| 81 | RG2833 | 17.84178312 | HDAC |
| 82 | UNC1215 | 17.60924571 | KME |
| 83 | Anacardic acid | 17.45834175 | Aurora Kinase |
| 84 | PFI-4 | 17.42723944 | Scaffold |
| 85 | RG108 | 17.35509271 | DNMT |
| 86 | XL228 | 17.29952174 | Aurora Kinase |
| 87 | EPZ015666 | 17.03484969 | HMT |
| 88 | SGI-1027 | 16.87448057 | DNMT |
| 89 | CI994 (Tacedinaline) | 16.46836602 | HDAC |
| 90 | BET bromodomain inhibitor | 16.33240381 | Bromodomain |
| 91 | TMP269 | 16.28616185 | HDAC |
| 92 | ME0328 | 15.81075477 | PARP |
| 93 | Fasudil (HA-1077) HCl | 15.80000583 | ROCK |
| 94 | Tubastatin A | 15.74772268 | HDAC |
| 95 | BI-2536 | 15.69341395 | PLK |
| 96 | OTX015 | 15.64914531 | BET |
| 97 | Dorsomorphin 2HCl | 15.62459204 | AMPK |
| 98 | 4SC-202 | 15.56860163 | HDAC |
| 99 | GLPG0634 | 15.52550086 | JAK |
| 100 | GSK126 | 15.47142806 | HMT |
| 101 | SP2509 | 15.43199934 | HDM |
| 102 | OF-1 | 15.37553038 | Bromodomain |
| 103 | Bromosporine | 15.35021806 | BET |
| 104 | EI1 | 15.27001984 | EZH2 |
| 105 | EPZ005687 | 15.1337905 | HMT |
| 106 | TG101209 | 15.10478372 | c-RET |
| 107 | CEP-33779 | 15.10174393 | JAK |
| 108 | GSK-LSD1 (hydrochloride) | 14.88238965 | HDM |
| 109 | N-Oxalylglycine | 14.35713603 | HDM |
| 110 | Go 6983 | 14.34122338 | PKC |
| 111 | BML-210(CAY10433) | 14.33025488 | HDAC |
| 112 | Mitomycin C | 14.13643371 | Apoptosis Inducers |
| 113 | CPI-203 | 14.05708296 | BET |
| 114 | Ellagic acid | 13.80252326 | Topoisomerase |
| 115 | ITF2357 (Givinostat) | 13.67204195 | HDAC |
| 116 | A-966492 | 13.59731593 | PARP |
| 117 | BAZ2-ICR | 13.5657073 | Bromodomain |
| 118 | GSK2879552 | 13.46654521 | Histone Demethylases |
| 119 | C-7280948 | 13.45430367 | PRMT |
| 120 | JIB-04 | 13.40345198 | HDM |
| 121 | Remodelin | 13.336818 | HAT |
| 122 | CPTH6 (hydrobromide) | 13.29776016 | HAT |
| 123 | Enzastaurin (LY317615) | 13.08461395 | PKC |
| 124 | Todralazine (hydrochloride) | 13.07077827 | HAT |
| 125 | Suberohydroxamic Acid | 12.98586621 | HDAC |
| 126 | PFI-3 | 12.92371722 | SMARC |
| 127 | Tasquinimod | 12.58392644 | HDAC |
| 128 | OICR-9429 | 12.58030593 | CRD |
| 129 | Belinostat (PXD101) | 12.54737644 | HDAC |
| 130 | HA-100 (hydrochloride) | 12.45439287 | Broad Spectrum Protein |
| | | | Kinase Inhibitor |
| 131 | KD 5170 | 12.42803589 | HDAC |
| 132 | Sirefungin | 12.42055751 | HMT |
| 133 | GSK2879552 | 12.3459145 | HDM |
| 134 | RVX-208 | 12.33294756 | BET |
| 135 | HDAC3 Inhibitor | 12.09113806 | HDAC |
| 136 | 6-Thioguanine | 12.09042871 | DNMT |
| 137 | CAY10591 | 11.91300227 | SIRT |
| 138 | A-366 | 11.91268455 | HMT |
| 139 | BAY 87-2243 | 11.79147668 | HIF |
| 140 | ORY-1001 | 11.72419514 | HDM |
| 141 | AZD 5153 | 11.51912795 | BET |
| 142 | CeMMEC13 | 11.5091713 | Bromodomain |
| 143 | IOX1 | 11.45346749 | HDM |
| 144 | Mitoxantrone HCl | 11.28031261 | Topoisomerase |
| 145 | AICAR | 11.26585175 | AMPK |
| 146 | NI-57 | 11.19521685 | Scaffold |
| 147 | BI-9564 | 10.99823425 | Scaffold |
| 148 | GSK 5959 | 10.99065668 | Bromodomain |
| 149 | EPZ004777 (formate) | 10.9101376 | HMT |
| 150 | 2,4-Pyridinedicarboxylic Acid (hydrate) | 10.85246523 | HDM |
| 151 | RGFP966 | 10.81127547 | HDAC |
| 152 | MK-8745 | 10.66356196 | Aurora Kinase |
| 153 | Sotrastaurin (AEB071) | 10.6256445 | PKC |
| 154 | MM-102 | 10.41204556 | HMT |
| 155 | AZD1208 | 10.38111747 | Pim |
| 156 | BRD73954 | 10.32748389 | HDAC |
| 157 | IOX2(Glycine) | 10.16628712 | HIF |
| 158 | Decitabine (NSC127716, 5AZA-CdR) | 10.10193767 | DNA Methyltransferase |
| 159 | WIKI4 | 10.07368247 | PARP |
| 160 | Fisetin | 10.0226773 | Sirtuin |
| 161 | Butyrolacetone 3 | 9.965622302 | HAT |
| 162 | EPZ020411 | 9.958495 | PRMT6 |
| 163 | Daminozide | 9.953755013 | HDM |
| 164 | PF-CBP1 hydrochloride | 9.952573738 | Bromodomain |
| 165 | Delphinidin (chloride) | 9.890317079 | HAT |
| 166 | Lomeguatrib | 9.850724141 | DNA Methyltransferase |
| 167 | Splitomicin | 9.669167751 | SIRT |
| 168 | PFI-1 | 9.636965011 | BET |
| 169 | AZD2461 | 9.59269325 | PARP |
| 170 | 5-Nitroso-8-quinolinol | 9.461680646 | HDAC |
| 171 | Mizoribine | 9.419444241 | IMPDH |
| 172 | JGB1741 | 9.246838213 | SIRT |
| 173 | 2′,3′,5′-triacetyl-5-azacytidine | 9.171043678 | DNMT |
| 174 | NCH 51 | 9.119422341 | HDAC |
| 175 | Thioguanine | 9.104309638 | DNA Methyltransferase |
| 176 | EPZ5676 | 9.087890208 | HMT |
| 177 | CCT137690 | 9.075906099 | Aurora Kinase |
| 178 | NSC 87877 | 8.877191038 | Protein Ser/Thr Phosphatases |
| 179 | MS436 | 8.851800269 | Bromodomain |
| 180 | Rucaparib (free base) | 8.810690654 | PARP |
| 181 | EPZ6438 | 8.683393972 | HMT |
| 182 | GSK591 | 8.604478418 | HMT |
| 183 | MI-2 | 8.541554796 | Menin-MLL |
| 184 | GSK-J1 (sodium salt) | 8.486936653 | HDM |
| 185 | CYC116 | 8.382960369 | Aurora Kinase |
| 186 | JIB-04 | 8.33959306 | Histone Demethylase |
| 187 | Aurora A Inhibitor I | 8.339195554 | Aurora Kinase |
| 188 | Gemcitabine HCl | 8.319275898 | DNA Synthesis |
| 189 | TC-H 106 | 8.223093865 | HDAC |
| 190 | CUDC-907 | 8.158791278 | HDAC |
| 191 | 3-Deazaneplanocin A (DZNep) hydrochloride | 8.135567809 | HMT |
| 192 | Dorsomorphin (Compourd C) | 7.913393323 | AMPK |
| 193 | AS8351 | 7.860897233 | Histone Demethylases |
| 194 | ML-324 | 7.483633324 | HDM |
| 195 | Baricitirib phosphate | 7.373299508 | JAK |
| 196 | HDAC 6 inhibitor | 7.32961506 | HDAC |
| 197 | Decernotinib(VX-509) | 7.32153635 | JAK |
| 198 | Phenformin HCl | 7.320359582 | Others |
| 199 | SGC-CBP30 | 7.287436549 | HAT |
| 200 | ITSA-1 (ITSA1) | 7.283230198 | HDAC |
| 201 | Staurosporine | 7.230393243 | Broad Spectrum Protein |
| | | | Kinase Inhibitor |
| 202 | UNC0224 | 7.132100641 | HMT |
| 203 | Decitabine | 7.089567928 | DNMT |
| 204 | RVX-208 | 7.058028545 | Bromodomain |
| 205 | EPZ015666 | 6.990590179 | PRMT |
| 206 | Cyproheptadine (hydrochloride hydrate) | 6.958181415 | HMT |
| 207 | MM-102 | 6.836983931 | HMT |
| 208 | EPZ004777 | 6.631516085 | HMT |
| 209 | MS023 (hydrochloride) | 6.531051839 | PRMT |
| 210 | Pirarubicin | 6.407710036 | Topoisomerase |
| 211 | Garcinol | 6.401455201 | HAT |
| 212 | Tenovin-1 | 6.377424813 | p53 |
| 213 | UNC 0642 | 6.253094287 | HMT |
| 214 | CPTH2 (hydrochloride) | 6.250555631 | HAT |
| 215 | KW 2449 | 6.231254445 | FLT3 |
| 216 | Sodium 4-phenylbutyrate | 6.103363726 | HDAC |
| 217 | Bardoxolone methyl | 6.087181275 | JAK |
| 218 | MI-2 (hydrochloride) | 6.017283433 | HMT |
| 219 | Ofloxacin | 5.913281265 | Topoisomerase |
| 220 | PX-478 2HCl | 5.84000741 | HIF |
| 221 | Coumarin | 5.809475914 | Immunology & Inflammation |
| | | | related |
| 222 | Diosgenin | 5.645849955 | STAT |
| 223 | I-BET-762 | 5.520411559 | Bromodomain |
| 224 | CI-Amidine (hydrochloride) | 5.451205447 | PAD |
| 225 | LFM-A13 | 5.441518058 | BTK |
| 226 | Anacardic Acid | 5.093475937 | HAT |
| 227 | PFI 3 | 5.077087994 | Bromodomain |
| 228 | SB1317 | 5.059012606 | JAK |
| 229 | Go 6976 | 5.045139424 | PKC |
| 230 | (−)-Epigallocatechin gallate (EGCG) | 4.980094683 | PKC |
| 231 | SGI-1027 | 4.876036815 | HMT |
| 232 | Doxorubicin | 4.863957523 | Topoisomerase |
| 233 | SRT1720 HCl | 4.809414002 | Sirtuin |
| 234 | NSC228155 | 4.767060421 | EGFR |
| 235 | ZM 449829 | 4.710953757 | JAK |
| 236 | ABC294640 | 4.656768505 | Sphingosine Kinase-2 |
| 237 | SGC707 | 4.518296984 | HMT |
| 238 | Romidepsin (FK229, depsipeptide) | 4.459035873 | HDAC |
| 239 | ML324 | 4.315092992 | Histone Demethylases |
| 240 | SGC0956 | 4.270648256 | PMT |
| 241 | Mirin | 4.239738886 | ATM/ATR |
| 242 | Zebularine | 4.20140482 | DNMT |
| 243 | BML-278 | 4.190747193 | SIRT |
| 244 | AMG-900 | 4.126409509 | Aurora Kinase |
| 245 | Plumbagin | 4.102003807 | STAT3, PLK1 |
| 246 | Procainamide HCl | 4.094949022 | Sodium Channel |
| 247 | CPI-203 | 4.089602712 | Bromodomain |
| 248 | EPZ5676 | 4.040495733 | HMT |
| 249 | C646 | 3.95755596 | HAT |
| 250 | (R)-(+)-Etomoxir sodium salt | 3.941264161 | HAT |
| 251 | CPI-169 | 3.906153686 | EZH2 |
| 252 | EPZ-6438 | 3.862127799 | HMT |
| 253 | 5-Azacytidine | 3.816790152 | DNMT |
| 254 | a-hydroxyglutaric Acid (sodium salt) | 3.785492136 | HDM |
| 255 | GF 109203X | 3.766318591 | PKC |
| 256 | Bromodomain Inhibitor, (+)-JQ1 | 3.74906995 | Bromodomain |
| 257 | PJ34 hydrochloride | 3.695750244 | PARP |
| 258 | A 366 | 3.679644867 | HMT |
| 259 | Tofacitinib (CP-690550) Citrate | 3.665607489 | JAK |
| 260 | Metformin HCl | 3.611644442 | Others |
| 261 | Olaparib (AZD2281, Ku-0059436) | 3.527001626 | PARP |
| 262 | TAK-901 | 3.351289493 | Aurora Kinase |
| 263 | Doxorubicin (Adriamycin) HCl | 3.259814011 | Topoisomerase |
| 264 | Daptomycin | 3.207390061 | DNA Synthesis |
| 265 | WP1066 | 3.078879172 | JAK |
| 266 | I-BET151 (GSK1210151A) | 3.071822685 | Bromocomain |
| 267 | Ro 31-8220 Mesylate | 3.047672556 | PKC |
| 268 | Cinnamic acid | 2.97083464 | Others |
| 269 | GSK-LSD1 2HCl | 2.922493079 | Histone Demethylases |
| 270 | D-erythro-Sphingosine (synthetic) | 2.713306438 | PKC |
| 271 | CCT129202 | 2.662187318 | Aurora Kinase |
| 272 | Ruxolitinib phosphate | 2.658885332 | JAK |
| 273 | Raddeanin A | 2.60882522 | HDAC |
| 274 | 2-hexyl-4-Pentynoic Acid | 2.598543713 | HDAC |
| 275 | Nanaomycin A | 2.594203209 | DNA Methyltransferase |
| 276 | PFI-1 (PF-6405761) | 2.510171593 | Bromocomain |
| 277 | Salvianolic acid B | 2.344983624 | Sirtuin |
| 278 | A-196 | 2.333831643 | HMT |
| 279 | OTX-015 | 2.250893992 | Bromodomain |
| 280 | IOX 1 | 2.197883905 | Histone Demethylases |
| 281 | PFI-2 (hydrochloride) | 2.188810376 | HMT |
| 282 | Sephin1 | 2.093926491 | Protein Ser/Thr Phosphatases |
| 283 | β-Glycerophosphate (sodium salt hydrate) | 2.069144546 | Protein Ser/Thr Phosphatases |
| 284 | WDR5 0103 | 1.920783632 | HMT |
| 285 | Cytarabine | 1.899670425 | DNA Synthesis |
| 286 | BMS-911543 | 1.849603222 | JAK |
| 287 | Tasquinimod | 1.848157809 | HDAC |
| 288 | Curcumin | 1.717863983 | KEAP1-Nrf2 |
| 289 | PRT4165 | 1.397531935 | E3 |
| 290 | GSK J1 | 1.380101251 | Histone Demethylases |
| 291 | SNS-314 Mesylate | 1.355220159 | Aurora Kinase |
| 292 | SGC 0946 | 1.267648697 | HMT |
| 293 | SP2509 | 1.235456008 | Histone Demethylases |
| 294 | Procarbazine HCl | 1.149481351 | DNA Synthesis |
| 295 | SGC-CBP30 | 0.99336391 | Bromodomain |
| 296 | Daprodustat(GSK1278863) | 0.971454279 | HIF |
| 297 | Resveratrol | 0.948183279 | Sirtuin |
| 298 | GSK126 | 0.822817655 | EZH2 |
| 299 | TC-E 5003 | 0.745290933 | PRMT |
| 300 | Tranylcypromine (hydrochloride) | 0.743727375 | HDM |
| 301 | TCS PIM-1 1 | 0.742373507 | Pim |
| 302 | C7280948 | 0.573474996 | HMT |
| 303 | GSK J2 | 0.570085433 | Histone Demethylases |
| 304 | Octyl-a-hydroxyglutarate | 0.558739623 | HDM |
| 305 | TMP-195 | 0.542463907 | HDAC |
| 306 | ZM 447439 | 0.508859042 | Aurora Kinase |
| 307 | Rucaparib (AG-014699, PF-01367338) | 0.494804362 | PARP |
| 308 | AZD1480 | 0.436104538 | JAK |
| 309 | UNC0321 (trifluoroacetate salt) | 0.427520591 | HMT |
| 310 | GSK484 (hydrochloride) | 0.315291593 | PAD4 |
| 311 | SBHA | 0.314649936 | HDAC |
| 312 | AG-14361 | 0.205857094 | PARP |
| 313 | GSK1324726A | 0.129466328 | Bromodomain |
| 314 | MK-5108 (VX-689) | 0.089923956 | Aurora Kinase |
| 315 | Tenovin-6 | 0.079333654 | Sirtuin |
| 316 | Cucurbitacin I | 0.023869326 | JAK |
| 317 | GSK503 | −0.098429729 | EZH2 |
| 318 | Amodiaquine dihydrochloride dihydrate | −0.119620724 | Transferase |
| 319 | BRD 7552 | −0.156457361 | Transcription Factors |
| 320 | FG-4592 (ASP1517) | −0.181643568 | HIF |
| 321 | (−)-JQ1 | −0.214815591 | Bromodomain |
| 322 | Myricetrin | −0.272294087 | PKC |
| 323 | Aurora Kinase Inhibitor III | −0.322608713 | Aurora Kinase |
| 324 | Inauhzin | −0.344068883 | Sirtuin |
| 325 | Pyridone 6 | −0.400789467 | JAK |
| 326 | Sodium butyrate | −0.446558886 | HDAC |
| 327 | MG 149 | −0.461429666 | HAT |
| 328 | PFI 4 | −0.540320639 | Bromodomain |
| 329 | C646 | −0.574550453 | HAT |
| 330 | EX 527 (SEN0014196) | −0.589211787 | Sirtuin |
| 331 | MS023 | −0.632290161 | HMT |
| 332 | Tranylcypromine hydrochloride | −0.634765724 | Histone Demethylases |
| 333 | MK-4827 | −0.755576148 | PARP |
| 334 | GSK343 | −0.823306355 | HMT |
| 335 | Thiomyristoyl | −0.838083301 | Sirtuin |
| 336 | Santacruzamate A (CAY10683) | −0.965273355 | HDAC |
| 337 | GSK-3 Inhibitor IX (BIO) | −1.008108971 | GSK-3 |
| 338 | ABT-888 (Veliparib) | −1.058456678 | PARP |
| 339 | LY2784544 | −1.068416855 | JAK |
| 340 | G007-LK | −1.130114592 | tankyrase |
| 341 | AT9283 | −1.133447497 | Aurora Kinase |
| 342 | SMI-4a | −1.184096388 | Pim |
| 343 | UNC 0224 | −1.296316589 | HMT |
| 344 | INO-1001 | −1.318675471 | PARP |
| 345 | Ginkgolide C | −1.318960735 | Others |
| 346 | EPZ020411 | −1.338061778 | PRMT |
| 347 | TG101348 (SAR302503) | −1.346337756 | JAK |
| 348 | 4-HQN | −1.369740231 | PARP |
| 349 | NVP-BSK805 | −1.371466644 | JAK |
| 350 | Cerdulatinib (PRT062070) | −1.40584687 | JAK |
| 351 | I-BET762 | −1.571538818 | BET |
| 352 | Danusertib (PHA-739358) | −1.71966924 | c-RET |
| 353 | Curcumol | −1.726205337 | JAK |
| 354 | WHI-P154 | −1.780434847 | JAK |
| 355 | Daminozide | −1.7811459 | HDAC |
| 356 | CeMMEC1 | −1.808787368 | Bromodomain |
| 357 | 4′-bromo-Resveratrol | −1.811106928 | SIRT |
| 358 | FG2216 | −1.815979115 | HIF |
| 359 | 2,4-Pyridinedicarboxylic Acid | −1.886938558 | Histone Demethylases |
| 360 | SRT2104 (GSK2245840) | −1.8998322 | Sirtuin |
| 361 | RN-1 (hydrochloride) | −2.198198784 | HDM |
| 362 | Pacritinib (SB1518) | −2.390543015 | FLT3, JAK |
| 363 | OG-L002 | −2.428874199 | Histone Demethylases |
| 364 | Nicotinamide | −2.471188994 | Sirtuin |
| 365 | BMN-673 8R, 9S | −2.488918271 | PARP |
| 366 | ETC-1002 | −2.506487604 | ATP citrate lyase |
| 367 | 12-O-tetradecanoyl phorbol-13-acetate (PMA) | −2.544880645 | PKC; SPHK |
| 368 | CPI-637 | −2.546061379 | Bromodomain |
| 369 | RG 108 | −2.614857148 | DNA Methyltransferase |
| 370 | PFI-2 | −2.630056496 | HMT |
| 371 | Baricitinib (LY3009104, INCB028050) | −2.640670751 | JAK |
| 372 | A-769662 | −2.693860882 | Others |
| 373 | Tofacitinib (CP-690550, Tasocitinib) | −2.780609639 | JAK |
| 374 | Sirtinol | −2.794907164 | Sirtuin |
| 375 | Veliparib dihydrochloride | −2.841189618 | PARP |
| 376 | Sulforaphane | −3.081547104 | KEAP1-Nrf2 |
| 377 | Valproic acid | −3.26791409 | HDAC |
| 378 | Splitomicin | −3.350135108 | Sirtuin |
| 379 | Parthenolide | −3.360194853 | HDAC |
| 380 | SirReal2 | −3.471683095 | Sirtuin |
| 381 | Zebularine | −3.497900813 | DNA Methyltransferase |
| 382 | Tenovin-6 (hydrochloride) | −3.503480524 | SIRT |
| 383 | KC7F2 | −3.552690191 | HIF |
| 384 | PTP Inhibitor I | −3.586177045 | Protein Ser/Thr Phosphatases |
| 385 | MS049 (hydrochloride) | −3.6536697 | PRMT |
| 386 | BI 2536 | −3.682092284 | PLK |
| 387 | UMB-32 | −3.691983503 | BET |
| 388 | HTH-01-015 | −3.923961896 | AMPK |
| 389 | CYT387 | −4.08701843 | JAK |
| 390 | Molidustat (BAY85-3934) | −4.286870753 | HIF |
| 391 | UNC669 | −4.299220979 | MBT |
| 392 | Ruxolitinib (INCB018424) | −4.311240896 | JAK |
| 393 | Flufenamic acid | −4.318210492 | AMPK; Calcium Channel; Chloride |
| | | | Channel; COX: Potassium Channel |
| 394 | UNC1215 | −4.35583064 | Bromodomain |
| 395 | BIX 01294 | −4.37190169 | HMT |
| 396 | Donepezil HCl | −4.516445142 | HAT |
| 397 | ZM 39923 HCl | −4.611416451 | JAK |
| 398 | PCI 34051 | −4.83396398 | HDAC |
| 399 | MC 1568 | −4.946551501 | HDAC |
| 400 | RGFP966 | −5.032821321 | HDAC |
| 401 | Methylstat (hydrate) | −5.079495934 | HDM |
| 402 | MCB-613 | −5.354590205 | HAT |
| 403 | Benzamide | −5.576444441 | PARP |
| 404 | UNC0642 | −5.587400991 | HMT |
| 405 | GSK2801 | −5.719020524 | Bromodomain |
| 406 | L-Sulforaphane | −5.740004094 | NRF2 |
| 407 | UNC669 | −5.867826313 | Bromodomain |
| 408 | XAV-939 | −6.03352837 | PARP |
| 409 | UPF 1069 | −6.051781139 | PARP |
| 410 | AR-42 (OSU-HDAC42) | −6.139770953 | HDAC |
| 411 | Divalproex Sodium | −6.451471205 | Autophagy |
| 412 | AZD1152 | −6.485464202 | Aurora Kinase |
| 413 | Quercetin | −6.523400052 | PI3K |
| 414 | JW 55 | −6.597518858 | PARP |
| 415 | 6-gingerol | −6.674462351 | Others |
| 416 | Hinokitiol | −6.717543158 | Others |
| 417 | Reversine | −6.768923787 | Aurora Kinase |
| 418 | Entacapone | −6.777144269 | HMT |
| 419 | GSK690693 | −7.029474973 | Akt |
| 420 | Tankyrase Inhibitors (TNKS) 49 | −7.40608522 | PARP |
| 421 | PX 12 | −7.518225671 | HIF |
| 422 | CX-6258 | −7.528335146 | Pim |
| 423 | Iniparib (BSI-201) | −7.529732031 | PARP |
| 424 | Sulforaphane | −7.657305653 | NRF2 |
| 425 | GLPG0634 analogue | −7.70189526 | JAK |
| 426 | MN 64 | −7.834691213 | tankyrase |
| 427 | L-a-Hydroxyglutaric Acid | −7.872830169 | HKD |
| 428 | BI-7273 | −8.336482416 | Bromodomain |
| 429 | DMOG | −8.654021523 | HIF |
| 430 | CPI-455 | −8.681627262 | Histone Demethylases |
| 431 | Latanoprost | −8.841913145 | Others |
| 432 | ORY-1001 | −9.011430375 | HDAC |
| 433 | UNC1999 | −9.67459369 | HMT |
| 434 | Midostaurin (PKC412) | −9.8909241 | PKC |
| 435 | GSK J4 HCl | −10.21199977 | Histone Demethylases |
| 436 | Bufexamac | −10.24828591 | COX |
| 437 | PTP Inhibitor II | −10.36302306 | Protein Ser/Thr Phosphatases |
| 438 | 5-Methyl-2′-deoxycytidine | −10.37220185 | Others |
| 439 | Bufexamac | −10.49925889 | HDAC |
| 440 | Nedaplatin | −10.66008865 | Adrenergic Receptor |
| 441 | AG-490 | −10.83481681 | EGFR |
| 442 | CAY10722 | −11.2016316 | SIRT |
| 443 | PHA-680632 | −11.27526425 | Aurora Kinase |
| 444 | BMN 673 | −11.28201903 | PARP |
| 445 | Triacetyl Resveratrol | −11.94142232 | Sirtuin |
| 446 | UNC0638 | −12.13191562 | HMT |
| 447 | TAK-632 | −12.17789346 | Raf |
| 448 | NVP-BSK805 2HCl | −12.66853892 | JAK |
| 449 | MLN8054 | −12.86594055 | Aurora Kinase |
| 450 | GSK-J4 (hydrochloride) | −13.06327703 | HDM |
| 451 | Carboplatin | −13.45575458 | DNA Synthesis |
| 452 | JANEX-1 | −13.67145532 | JAK |
| 453 | Salermide | −13.8765124 | SIRT |
| 454 | 1,2,3,4,5,6-Hexabromocyclohexane | −13.97992187 | JAK |
| 455 | XL019 | −14.17955887 | JAK |
| 456 | Sirtinol | −14.60770988 | SIRT |
| 457 | LLY507 | −14.82100313 | HMT |
| 458 | JNJ-7706621 | −15.24023004 | Aurora Kinase |
| 459 | GSK343 | −15.40049757 | EZH2 |
| 460 | Tubacin | −15.59003335 | HDAC |
| 461 | Sal 003 | −16.27870041 | Protein Ser/Thr Phosphatases |
| 462 | Barasertib (AZD1152-HQPA) | −16.49490381 | Aurora Kinase |
| 463 | SGI-1776 free base | −17.3729288 | Pim |
| 464 | MC1568 | −17.77566005 | HDAC |
| 465 | Chelerythrine | −18.61508402 | PKC |
| 466 | Sodium Phenylbutyrate | −19.07418797 | HDAC |
| 467 | Valproic acid sodium salt (Sodium valproate) | −19.3577127 | HDAC |
| 468 | VX-680 (MK-0457, Tozasertib) | −20.25477484 | Aurora Kinase |
| 469 | Chelerythrine Chloride | −21.49677635 | PKC |
| 470 | Droxinostat | −21.53261779 | HDAC |
| 471 | PCI-34051 | −22.68211701 | HDAC |
| 472 | ENMD-2076 | −22.79705863 | Aurora Kinase |
| 473 | ENMD-2076 L-(+)-Tartaric acid | −24.46111835 | Aurora Kinase |
| 474 | WZ4003 | −24.50613794 | AMPK |
| 475 | HAT Inhibitor II | −25.45786815 | HAT |
| 476 | MLN8237 (Alisertib) | −25.50541945 | Aurora Kinase |
| 477 | UNC1999 | −26.21583659 | HMT |
| 478 | UNC0636 | −26.35492189 | HMT |
| 479 | L002 | −39.83333226 | HAT |
| 480 | PBIT | −46.18508938 | HDM |

### Example 6: HDAC Inhibition is More Effective with MYC and HSF1 Dual Amplification

Results of this screen indicated 45 of the top 54 hits were compounds that targeted histone deacetylases (HDACs). (Table 1). To test the effect of HDAC inhibitors on cell lines having dual copy number gain in both MYC and HSF1, we treated OVCAR8 (positive for MYC-HSF1 amplification) for 24 hrs with an HDAC inhibitor, etinostat. Total RNA from treated cells was subjected to RTqPCR. To conduct the RTqPCR, RNA was extracted from cells using an RNA isolation kit with DNAse treatment (Zymo). RNA was subjected to reverse transcription using RT Master Mix (Applied Biosystems). qPCR was performed with SYBR Green Master Mix (Applied Biosystems) using a QuantStudio3 (Applied Biosystems). Experiments were performed in biological triplicate and analyzed using the AACt method.

Entinostat, an HDAC inihibitor, significantly reduced levels of HSF1 and MYC target genes (FIG. 6B), indicating that entinostat reduces activity of MYC and HSF1 transcription. Entinostat was also observed to reduced protein levels of both MYC and HSF1 (FIG. 6C and FIG. 6D). Immunoblotting and co-immunoprecipitation was performed as described above. Antibodies for immunoblotting and immunoprecipitation included MYC (CST), HSF1 (CST), GAPDH (CST). The reduced protein levels of both MYC and HSF1 after treatment indicated that etinostat is likely to contribute to the decreased transcriptional activity of MYC and HSF1 (FIG. 6B). Through the innovative method of screening potential therapeutics against cells having dual amplification of MYC and HSF1, Applicant was able to identify potential therapeutic targets, HDAC inhibitors, and therapeutics having increased efficacy against these cancer cells.

### General Methods and Materials Used for Examples

Cell Culture and Reagents: All cell lines were purchased from ATCC and cultured in ATCC recommended culture media at 37° C. with 5% CO2. All reagents were purchased from Fisher Scientific unless otherwise noted. siRNA were purchased from Bioneer. Volasertib was purchased from Cayman Chemical.

Immunoblotting and Immunoprecipitation: Immunoblotting and co-immunoprecipitation was performed as we have previously described (Lu et al., bioRxiv 2020:2020.08.31.275909). Antibodies for immunoblotting and immunoprecipitation included MYC (CST), HSF1 (CST), β-actin (CST), GAPDH (CST), FLAG(Sigma), and p-HSF1 (S326) (Abcam).

Luciferase Reporter Assays: Luciferase reporter assays were performed as we previously described (Lu et al. bioRxiv 2020:2020.08.31.275909). The HSF1 activity reporter contains multiple heat shock element (HSE) motifs driving firefly luciferase. Experiments were performed with co-transfection of a constitutively active renilla reporter. Analysis was completed by dividing firefly activity by renilla activity.

Immunohistochemistry: Tissues were subjected to immunohistochemistry as we previously described (Carpenter et al. Oncotarget 2017; 8:73947-63). Briefly, slides were deparaffinized and rehydrated prior to antigen retrieval using heat and pressure. Slides had endogenous peroxidase activity blocked with Bloxall (VectorLabs) and signal developed with DAB (Vector Labs). Antibodies used for IHC included MYC (Santa Cruz), p-HSF1 (S326) (Abcam), HSF1 (CST), p-PLK1 (T210) (CST). Slides were imaged with Motic Easy Scan and analyzed with QuPath.

### Equivalents and Scope

Those skilled in the art will recognize or be able to ascertain using no more than routine experimentation many equivalents to the specific embodiments described herein. The scope of the present invention is not intended to be limited to the above, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, it is to be understood that the invention encompasses all variations, combinations, and permutations in which one or more limitations, elements, clauses and descriptive terms, from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should be understood that, in general, where the invention, or aspects of the invention is/are referred to as comprising particular elements, features, etc., certain embodiments of the invention or aspects of the invention consist, or consist essentially of, such elements, features, etc. For purposes of simplicity, those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranged can assume any specific value or sub-range within the stated ranges in different embodiments of the invention, to the tenth of the unit of the lower limit of the range, unless the context clearly dictates otherwise.

The term “about” or “approximately” means within an acceptable error range for the particular value as determined by one of the ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5% or up to 1% of a given value. Alternatively, the term can mean within an order of magnitude, for example within 5-fold, or within 2-fold, of a value. Where particular values are described in the application and claims, unless otherwise stated the term “about” meaning within an acceptable error range for the particular value should be assumed.

In addition, it is to be understood that any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the method of the invention can be excluded from any one or more claims, for any reason, whether or not related to the existence of prior art.

### REFERENCES

Each of the patents, patent applications and references set out in this disclosure is hereby incorporated by reference, particularly for the teaching specifically referenced and/or discussed herein.
1. ACS. Cancer Facts & FIGS. 2020. Atlanta, GA2020;
2. Siegel R L, Miller K D, Jemal A. Cancer statistics, 2019. C A Cancer J Clin 2019; 69:7-34;
3. Lengyel E. Ovarian cancer development and metastasis. The American journal of pathology 2010; 177:1053-64;
4. Bowtell D D, Bohm S, Ahmed A A, Aspuria P J, Bast R C, Jr., Beral V et al. Rethinking ovarian cancer I I: reducing mortality from high-grade serous ovarian cancer. Nat Rev Cancer 2015; 15:668-79;
5. Cress R D, Chen Y S, Morris C R, Petersen M, Leiserowitz G S. Characteristics of Long-Term Survivors of Epithelial Ovarian Cancer. Obstet Gynecol 2015; 126:491-7;
6. Labidi-Galy S I, Papp E, Hallberg D, Niknafs N, Adleff V, Noe M, et al. High grade serous ovarian carcinomas originate in the fallopian tube. Nat Commun 2017; 8:1093;
7. Lheureux S, Gourley C, Vergote I, Oza A M. Epithelial ovarian cancer. Lancet 2019; 393:1240-53;
8. Torre L A, Trabert B, DeSantis C E, Miller K D, Samimi G, Runowicz C D, et al. Ovarian cancer statistics, 2018. C A Cancer J Clin 2018; 68:284-96;
9. Chuaqui R F, Cole K A, Emmert-Buck M R, Merino M J. Histopathology and molecular biology of ovarian epithelial tumors. Ann Diagn Pathol 1998; 2:195-207;
10. Chen C H, Shen J, Lee W J, Chow S N. Overexpression of cyclin D1 and c-Myc gene products in human primary epithelial ovarian cancer. Int J Gynecol Cancer 2005; 15:878-83;
11. Baker V V, Borst M P, Dixon D, Hatch K D, Shingleton H M, Miller D. c-myc amplification in ovarian cancer. Gynecol Oncol 1990; 38:340-2;
12. Buchynska L G, Brieieva O V, Iurchenko N P. Assessment of HER-2/neu, small es, Cyrillic-MYC and CCNEl gene copy number variations and protein expression in endometrial carcinomas. Exp Oncol 2019; 41:138-43;
13. Jung M, Russell A J, Kennedy C, Gifford A J, Mallitt K A, Sivarajasingam S, et al. Clinical Importance of Myc Family Oncogene Aberrations in Epithelial Ovarian Cancer. JNCI Cancer Spectr 2018; 2:pky047;
14. Li C, Bonazzoli E, Bellone S, Choi J, Dong W, Menderes G, et al. Mutational landscape of primary, metastatic, and recurrent ovarian cancer reveals c-MYC gains as potential target for BET inhibitors. Proceedings of the National Academy of Sciences of the United States of America 2019; 116:619-24;
15. Papp E, Hallberg D, Konecny G E, Bruhm D C, Adleff V, Noe M, et al. Integrated Genomic, Epigenomic, and Expression Analyses of Ovarian Cancer Cell Lines. Cell Rep 2018; 25:2617-33;
16. Tian X, Song J, Zhang X, Yan M, Wang S, Wang Y, et al. MYC-regulated pseudogene HMGA1P6 promotes ovarian cancer malignancy via augmenting the oncogenic HMGA1/2. Cell Death Dis 2020; 11:167;
17. CGARN. Integrated genomic analyses of ovarian carcinoma. Nature 2011; 474:609-15;
18. Iba T, Kigawa J, Kanamori Y, Itamochi H, Oishi T, Simada M, et al. Expression of the c-myc gene as a predictor of chemotherapy response and a prognostic factor in patients with ovarian cancer. Cancer Sci 2004; 95:418-23;
19. Colombo I, Garg S, Danesh A, Bruce J, Shaw P, Tan Q, et al. Heterogeneous alteration of the ERBB3-MYC axis associated with MEK inhibitor resistance in a KRAS-mutated low-grade serous ovcancer patient. Cold Spring Harb Mol Case Stud 2019; 5;
20. Sheng Q, Zhang Y, Wang Z, Ding J, Song Y, Zhao W. Cisplatin-mediated down-regulation of miR-145 contributes to up-regulation of P D-L1 via the c-Myc transcription factor in cisplatin-resistant ovarian carcinoma cells. Clin Exp Immunol 2020; 200:45-52;
21. Yi J, Liu C, Tao Z, Wang M, Jia Y, Sang X, et al. MYC status as a determinant of synergistic response to Olaparib and Palbociclib in ovarian cancer. EBioMedicine 2019; 43:225-37;
22. Zeng M, Kwiatkowski N P, Zhang T, Nabet B, Xu M, Liang Y, et al. Targeting MYC dependency in ovarian cancer through inhibition of CDK7 and CDK12/13. Elife 2018; 7;
23. Meyer N, Penn L Z. Reflecting on 25 years with MYC. Nat Rev Cancer 2008; 8:976-90;
24. Dudler R, Travers A A. Upstream elements necessary for optimal function of the hsp 70 promoter in transformed flies. Cell 1984; 38:391-8;
25. Parker C S, Topol J. A Drosophila RNA polymerase I I transcription factor contains a promoterregion-specific DNA-binding activity. Cell 1984; 36:357-69;
26. Topol J, Ruden D M, Parker C S. Sequences required for in vitro transcriptional activation of a Drosophila hsp 70 gene. Cell 1985; 42:527-37;
27. Gomez-Pastor R, Burchfiel E T, Thiele D J. Regulation of heat shock transcription factors and their roles in physiology and disease. Nat Rev Mol Cell Biol 2018; 19:4-19;
28. Cen H, Zheng S, Fang Y M, Tang X P, Dong Q. Induction of HSF1 expression is associated with sporadic colorectal cancer. World journal of gastroenterology 2004; 10:3122-6;
29. Chen Y F, Wang S Y, Yang Y H, Zheng J, Liu T, Wang L. Targeting HSF1 leads to an antitumor effect in human epithelial ovarian cancer. Int J Mol Med 2017; 39:1564-70;
30. Cheng Q, Chang J T, Geradts J, Neckers L M, Haystead T, Spector N L, et al. Amplification and high level expression of heat shock protein 90 marks aggressive phenotypes of human epidermal growth factor receptor 2 negative breast cancer. Breast cancer research: BCR 2012; 14:R62;
31. Chuma M, Sakamoto N, Nakai A, Hige S, Nakanishi M, Natsuizaka M, et al. Heat shock factor 1 accelerates hepatocellular carcinoma development by activating nuclear factor kappa B/mitogen-activated protein kinase. Carcinogenesis 2014; 35:272-81;
32. Cui J, Tian H, Chen G. Upregulation of Nuclear Heat Shock Factor 1 Contributes to Tumor Angiogenesis and Poor Survival in Patients With Non-Small Cell Lung Cancer. The Annals of thoracic surgery 2015; 100:465-72;
33. Dai C, Whitesell L, Rogers A B, Lindquist S. Heat shock factor 1 is a powerful multifaceted modifier of carcinogenesis. Cell 2007; 130:1005-18;
34. Dudeja V, Chugh R K, Sangwan V, Skube S J, Mujumdar N R, Antonoff M B, et al. Prosurvival role of heat shock factor 1 in the pathogenesis of pancreatobiliary tumors. American journal of physiology Gastrointestinal and liver physiology 2011; 300:G948-55;
35. Engerud H, Tangen I L, Berg A, Kusonmano K, Halle M K, Oyan A M, et al. High level of HSF1 associates with aggressive endometrial carcinoma and suggests potential for HSP90 inhibitors. British journal of cancer 2014; 111:78-84;
36. Fang F, Chang R, Yang L. Heat shock factor 1 promotes invasion and metastasis of hepatocellular carcinoma in vitro and in vivo. Cancer 2012; 118:1782-94;
37. Hoang A T, Huang J, Rudra-Ganguly N, Zheng J, Powell W C, Rabindran S K, et al. A novel association between the human heat shock transcription factor 1 (HSF1) and prostate adenocarcinoma. The American journal of pathology 2000; 156:857-64;
38. Ishiwata J, Kasamatsu A, Sakuma K, Iyoda M, Yamatoji M, Usukura K, et al. State of heat shock factor 1 expression as a putative diagnostic marker for oral squamous cell carcinoma. International journal of oncology 2012; 40:47-52;
39. Jego G, Lanneau D, De Thonel A, Berthenet K, Hazoume A, Droin N, et al. Dual regulation of SPI1/P U.1 transcription factor by heat shock factor 1 (HSF1) during macrophage differentiation of monocytes. Leukemia 2014; 28:1676-86;
40. Jin X, Moskophidis D, Mivechi N F. Heat shock transcription factor 1 is a key determinant of HCC development by regulating hepatic steatosis and metabolic syndrome. Cell metabolism 2011; 14:91-103;
41. Kim S A, Kwon S M, Yoon J H, Ahn S G. The antitumor effect of PLK1 and HSF1 double knockdown on human oral carcinoma cells. International journal of oncology 2010; 36:867-72;
42. Kourtis N, Moubarak R S, Aranda-Orgilles B, Lui K, Aydin I T, Trimarchi T, et al. FBXW7 modulates cellular stress response and metastatic potential through HSF1 post-translational modification. Nature cell biology 2015; 17:322-32;
43. Li S, Ma W, Fei T, Lou Q, Zhang Y, Cui X, et al. Upregulation of heat shock factor 1 transcription activity is associated with hepatocellular carcinoma progression. Molecular medicine reports 2014; 10:2313-21;
44. Mendillo M L, Santagata S, Koeva M, Bell G W, Hu R, Tamimi R M, et al. HSF1 drives a transcriptional program distinct from heat shock to support highly malignant human cancers. Cell 2012; 150:549-62;
45. Min J N, Huang L, Zimonjic D B, Moskophidis D, Mivechi N F. Selective suppression of lymphomas by functional loss of Hsf1 in a p53-deficient mouse model for spontaneous tumors. Oncogene 2007; 26:5086-97;
46. Nakamura Y, Fujimoto M, Hayashida N, Takii R, Nakai A, Muto M. Silencing HSF1 by short hairpin RNA decreases cell proliferation and enhances sensitivity to hyperthermia in human melanoma cell lines. Journal of dermatological science 2010; 60:187-92;
47. Powell C D, Paullin T R, Aoisa C, Menzie C J, Ubaldini A, Westerheide S D. The Heat Shock Transcription Factor HSF1 Induces Ovarian Cancer Epithelial-Mesenchymal Transition in a 3D Spheroid Growth Model. PLOS One 2016; 11:e0168389;
48. Santagata S, Hu R, Lin N U, Mendillo M L, Collins L C, Hankinson S E, et al. High levels of nuclear heat-shock factor 1 (HSF1) are associated with poor prognosis in breast cancer. Proceedings of the National Academy of Sciences of the United States of America 2011; 108:18378-83;
49. Tsukao Y, Yamasaki M, Miyazaki Y, Makino T, Takahashi T, Kurokawa Y, et al. Overexpression of heat-shock factor 1 is associated with a poor prognosis in esophageal squamous cell carcinoma. Oncology letters 2017; 13:1819-25;
50. Wu P S, Chang Y H, Pan C C. High expression of heat shock proteins and heat shock factor-1 distinguishes an aggressive subset of clear cell renal cell carcinoma. Histopathology 2017; 71:711-8;
51. Yasuda K, Hirohashi Y, Mariya T, Murai A, Tabuchi Y, Kuroda T, et al. Phosphorylation of HSF1 at serine 326 residue is related to the maintenance of gynecologic cancer stem cells through expression of HSP27. Oncotarget 2017; 8:31540-53;
52. Zhang N, Wu Y, Lyu X, Li B, Yan X, Xiong H, et al. HSF1 upregulates ATG4B expression and enhances epirubicin-induced protective autophagy in hepatocellular carcinoma cells. Cancer letters 2017; 409:81-90;
53. Zhou Z, Li Y, Jia Q, Wang Z, Wang X, Hu J, et al. Heat shock transcription factor 1 promotes the proliferation, migration and invasion of osteosarcoma cells. Cell proliferation 2017; 50;
54. Carpenter R L, Sirkisoon S, Zhu D, Rimkus T, Harrison A, Anderson A, et al. Combined inhibition of AKT and HSF1 suppresses breast cancer stem cells and tumor growth. Oncotarget 2017; 8:73947-63;
55. Gökmen-Polar Y, Badve S. Upregulation of HSF1 in estrogen receptor positive breast cancer. Oncotarget 2016; 7:84239-45;
56. Choi B H, Pagano M, Dai W. Plk1 protein phosphorylates phosphatase and tensin homolog (PTEN) and regulates its mitotic activity during the cell cycle. J Biol Chem 2014; 289:14066-74;
57. Lee Y J, Kim E H, Lee J S, Jeoung D, Bae S, Kwon S H, et al. HSF1 as a mitotic regulator: phosphorylation of HSF1 by Plk1 is essential for mitotic progression. Cancer Res 2008; 68:7550-60;
58. Li Z, Li J, Bi P, Lu Y, Burcham G, Elzey B D, et al. Plk1 phosphorylation of PTEN causes a tumor promoting metabolic state. Mol Cell Biol 2014; 34:3642-61;
59. Liu X S, Song B, Elzey B D, Ratliff T L, Konieczny S F, Cheng L, et al. Polo-like kinase 1 facilitates loss of Pten tumor suppressor-induced prostate cancer formation. J Biol Chem 2011; 286:35795-800;
60. Ren Y, Bi C, Zhao X, Lwin T, Wang C, Yuan J, et al. PLK1 stabilizes a MYC-dependent kinase network in aggressive B cell lymphomas. J Clin Invest 2018; 128:5517-30;
61. Hamanaka R, Maloid S, Smith M R, O'Connell C D, Longo D L, Ferris D K. Cloning and characterization of human and murine homologues of the Drosophila polo serine-threonine kinase. Cell Growth Differ 1994; 5:249-57;
62. Kressin M, Fietz D, Becker S, Strebhardt K. Modelling the Functions of Polo-Like Kinases in Mice and Their Applications as Cancer Targets with a Special Focus on Ovarian Cancer. Cells 2021; 10;
63. Zitouni S, Nabais C, Jana S C, Guerrero A, Bettencourt-Dias M. Polo-like kinases: structural variations lead to multiple functions. Nature reviews Molecular cell biology 2014; 15:433-52;
64. Liu Z, Sun Q, Wang X. PLK1, A Potential Target for Cancer Therapy. Transl Oncol 2017; 10:22-32;
65. Raab M, Sanhaji M, Zhou S, Rödel F, El-Balat A, Becker S, et al. Blocking Mitotic Exit of Ovarian Cancer Cells by Pharmaceutical Inhibition of the Anaphase-Promoting Complex Reduces Chromosomal Instability. Neoplasia 2019; 21:363-75;
66. Rödel F, Zhou S, Györffy B, Raab M, Sanhaji M, Mandal R, et al. The Prognostic Relevance of the Proliferation Markers Ki-67 and Plk1 in Early-Stage Ovarian Cancer Patients With Serous, Low-Grade Carcinoma Based on mRNA and Protein Expression. Front Oncol 2020; 10:558932;
67. Takai N, Yoshimatsu J, Nishida Y, Narahara H, Miyakawa I, Hamanaka R. Expression of polo-like kinase (PLK) in the mouse placenta and ovary. Reprod Fertil Dev 1999; 11:31-5;
68. Weichert W, Denkert C, Schmidt M, Gekeler V, Wolf G, Köbel M, et al. Polo-like kinase isoform expression is a prognostic factor in ovarian carcinoma. British journal of cancer 2004; 90:815-21;
69. Zhang H, Zhang K, Xu Z, Chen Z, Wang Q, Wang C, et al. MicroRNA-545 suppresses progression of ovarian cancer through mediating PLK1 expression by a direct binding and an indirect regulation involving KDM4B-mediated demethylation. BMC Cancer 2021; 21:163;
70. Zhang R, Shi H, Ren F, Liu H, Zhang M, Deng Y, et al. Misregulation of polo-like protein kinase 1, P53 and P21WAF1 in epithelial ovarian cancer suggests poor prognosis. Oncol Rep 2015; 33:1235-42;
71. Zhang S, Jing Y, Zhang M, Zhang Z, Ma P, Peng H, et al. Stroma-associated master regulators of molecular subtypes predict patient prognosis in ovarian cancer. Sci Rep 2015; 5:16066;
72. Gatza M L, Lucas J E, Barry W T, Kim J W, Wang Q, Crawford M D, et al. A pathway-based classification of human breast cancer. Proceedings of the National Academy of Sciences of the United States of America 2010; 107:6994-9;
73. Jacobs C, Shah S, Lu W-C, Ray H, Wang J, Nephew K P, et al. HSF1 excludes CD8+T cells from breast tumors via suppression of CCL5. bioRxiv 2022:2022.05.12.491688;
74. Lu W-C, Omari R, Ray H, Carpenter R L. AKT1 mediates multiple phosphorylation events that functionally promote HSF1 activation. bioRxiv 2020:2020.08.31.275909;
75. Wang W, Fang F, Ozes A, Nephew K P. Targeting Ovarian Cancer Stem Cells by Dual Inhibition of HOTAIR and DNA Methylation. Mol Cancer Ther 2021;
76. Xu M, Lin L, Chuang K-H, Ram B, Dai S, Su K-H, et al. Heat Shock Factor 1 (HSF1) specifically potentiates c-MYC-mediated transcription independently of the canonical heat-shock response. bioRxiv 2022:2022.02.22.481519;
77. Carpenter R L, Paw I, Dewhirst M W, Lo H W. Akt phosphorylates and activates HSF-1 independent of heat shock, leading to Slug overexpression and epithelial-mesenchymal transition (EMT) of HER2-overexpressing breast cancer cells. Oncogene 2015; 34:546-57;
78. Rudolph D, Steegmaier M, Hoffmann M, Grauert M, Baum A, Quant J, et al. B I 6727, a Polo-like kinase inhibitor with improved pharmacokinetic profile and broad antitumor activity. Clin Cancer Res 2009; 15:3094-102;
79. Pujade-Lauraine E, Selle F, Weber B, Ray-Coquard I L, Vergote I, Sufliarsky J, et al. Volasertib Versus Chemotherapy in Platinum-Resistant or Refractory Ovarian Cancer: A

Randomized Phase I I Groupe des Investigateurs Nationaux pour l′Etude des Cancers de l'Ovaire

Study. J Clin Oncol 2016; 34:706-13;
80. Xie F F, Pan S S, Ou R Y, Zheng Z Z, Huang X X, Jian M T, et al. Volasertib suppresses tumor growth and potentiates the activity of cisplatin in cervical cancer. Am J Cancer Res 2015; 5:3548-59;
81. Cigliano A, Pilo M G, Li L, Latte G, Szydlowska M, Simile M M, et al. Deregulated c-Myc requires a functional HSF1 for experimental and human hepatocarcinogenesis. Oncotarget 2017; 8:90638-50;
82. Zhu W, Niu J, He M, Zhang L, Lv X, Liu F, et al. SNORD89 promotes stemness phenotype of ovarian cancer cells by regulating Notch1-c-Myc pathway. J Transl Med 2019; 17:259;
83. Spänkuch B, Matthess Y, Knecht R, Zimmer B, Kaufmann M, Strebhardt K. Cancer inhibition in nude mice after systemic application of U6 promoter-driven short hairpin RNAs against PLK1. J Natl Cancer Inst 2004; 96:862-72;
84. Spänkuch-Schmitt B, Bereiter-Hahn J, Kaufmann M, Strebhardt K. Effect of RNA silencing of pololike kinase-1 (PLK1) on apoptosis and spindle formation in human cancer cells. J Natl Cancer Inst 2002; 94:1863-77;
85. Spänkuch-Schmitt B, Wolf G, Solbach C, Loibl S, Knecht R, Stegmüller M, et al. Downregulation of human polo-like kinase activity by antisense oligonucleotides induces growth inhibition in cancer cells. Oncogene 2002; 21:3162-71;
86. Lu W-C, Omari R, Ray H, Carpenter R L. AKT1 mediates multiple phosphorylation events that functionally promote HSF1 activation. bioRxiv 2020:2020.08.31.275909.

I claim:

1. A method of determining whether a biological sample obtained from a human has increased susceptibility to a therapeutic inhibitor comprising:
a) measuring copy number of the MYC gene in the biological sample and determining whether or not the MYC gene has a copy number of greater than or equal to three; and
b) measuring copy number of the HSF1 gene in the biological sample and determining whether or not the HSF1 gene has a copy number of greater than or equal to three,
wherein determining the copy number of greater than or equal to three for the MYC gene and the HSF1 gene indicates increased susceptibility to the therapeutic inhibitor.

2. The method of claim 1, wherein the biological sample is a tumor.

3. The method of claim 2, wherein the tumor comprises ovarian cancer cells.

4. The method of claim 3, wherein determining a copy number of the MYC gene of greater than or equal to five and a copy number of the HSF1 gene of greater than or equal to five in at least 5% of the ovarian cancer cells in the biological sample indicates increased susceptibility to the therapeutic inhibitor.

5. The method of claim 1, wherein measuring copy number comprises analyzing the biological sample with fluorescence in situ hybridization, comparative genomic hybridization, polymerase chain reaction, next-generation sequencing, southern blot analysis, immunohistochemistry, or a combination thereof.

6. The method of claim 1, wherein the therapeutic inhibitor is a PLK1 inhibitor or an HDAC inhibitor.

7. The method of claim 6, wherein the HDAC inhibitor is entinostat, vorinostat, romidepsin, panobinostat, or belinostat; and the PLK1 inhibitor is volasertib, BI2536, BI6727, NMS-1286937, or GSK461364.

8. A method for screening an epigenetic inhibitor against a biological sample comprising cancer cells, wherein at least 5% of the cancer cells comprise greater than or equal to three gene copies of MYC and greater than or equal to three gene copies of HSF1, comprising:
a) contacting the biological sample with the epigenetic inhibitor;
b) measuring average cell viability of the biological sample following contact with the epigenetic inhibitor; and
c) determining whether the biological sample has reduced average cell viability following contact with the epigenetic inhibitor relative to an untreated portion of the biological sample,
wherein reduced average cell viability indicates increased susceptibility to the epigenetic inhibitor.

9. The method of claim 8, wherein average cell viability is measured using a dye exclusion assay, a colorimetric assay, a fluorometric assay, a luminometric assay, or a flow cytometric assay.

10. The method of claim 9, wherein the biological sample comprises prostate cancer cells, bladder cancer cells, breast cancer cells, ovarian cancer cells, colorectal cancer cells, lung cancer cells, or esophageal cancer cells.

11. The method of claim 8, wherein the epigenetic inhibitor is an HDAC inhibitor.

12. The method of claim 8, comprising contacting the biological sample with the epigenetic inhibitor and a PLK-1 inhibitor.

13. The method of claim 12, wherein the PLK-1 inhibitor is volasertib, BI2536, BI6727, NMS-1286937, or GSK461364.

14. A method of treating a cancer in a mammalian subject comprising administering a therapeutically effective amount of an inhibitor to the subject, wherein at least one cell in a sample of cancer cells obtained from the mammalian subject has greater than or equal to three gene copies of MYC and greater than or equal to three gene copies of HSF1.

15. The method of claim 14, wherein the cancer is ovarian cancer, prostate cancer, bladder cancer, breast cancer, ovarian cancer, colorectal cancer, lung cancer, or esophageal cancer.

16. The method of claim 14, wherein the inhibitor is a PLK1 inhibitor.

17. The method of claim 16, wherein the PLK1 inhibitor is volasertib, BI2536, BI6727, NMS-1286937, or GSK461364.

18. The method of claim 14, wherein the inhibitor is an HDAC inhibitor.

19. The method of claim 18, wherein the HDAC inhibitor is entinostat, vorinostat, romidepsin, panobinostat, or belinostat.

20. The method of claim 14, wherein at least 5% of cells in the sample of cancer cells obtained from the subject have greater than or equal to five gene copies of MYC and greater than or equal to five gene copies of HSF1.
